(19) United States
(12) Patent Application Publication
(10) Pub. No.: US 2010/0178653 A1
(43) Pub. Date: Jul. 15, 2010

(54) GENE EXPRESSION SIGNATURE FOR CLASSIFICATION OF CANCERS

(75) Inventors: Ranit Aharonov, Tel Aviv (IL); Nitzan Rosenfeld, Rehovot (IL); Shai Rosenwald, Nes Ziona (IL); Iris Barshack, Tel Aviv (IL)

Correspondence Address: FOLEY AND LARDNER LLP;SUITE 500, 3000 K STREET NW, WASHINGTON, DC, 20007, US

(73) Assignees: Rosetta Genomics LTD.; TEL HASHOMER MEDICAL INFRASTRUCTURE AND SERVICES LTD.

(21) Appl. No.: 12/532,940

(22) PCT Filed: Mar. 20, 2008
(86) PCT No.: PCT/IL2008/000396

§ 371 Date: Sep. 24, 2009

Related U.S. Application Data
(60) Provisional application No. 60/907,266, filed on Mar. 27, 2007.
(60) Provisional application No. 60/929,244, filed on Jun. 19, 2007.
(60) Provisional application No. 61/024,565, filed on Jan. 30, 2008.

(51) Int. Cl. C12Q 1/68

(52) U.S. Cl. 435/6

(57) ABSTRACT

The present invention provides a process for classification of cancers and tissues of origin through the analysis of the expression patterns of specific microRNAs and nucleic acid molecules relating thereto. Classification according to a microRNA tree-based expression framework allows optimization of treatment, and determination of specific therapy.

### FIELD OF THE INVENTION

The present invention relates to methods for classification of cancers and the identification of their tissues of origin. Specifically the invention relates to microRNA molecules associated with specific cancers, as well as various nucleic acid molecules relating thereto or derived therefrom.

### BACKGROUND OF THE INVENTION

microRNAs are a novel class of non-coding, regulatory RNA genes¹⁻³ which are involved in oncogenesis⁴ and show remarkable tissue-specificity⁵⁻⁷. They have emerged as highly tissue-specific biomarkers^{2,5,6} postulated to play important roles in encoding developmental decisions of differentiation. Various studies have tied microRNAs to the development of specific malignancies⁴.

Metastatic cancer of unknown primary (CUP) accounts for 3-5% of all new cancer cases, and as a group is usually a very aggressive disease with a poor prognosis¹⁰. The concept of CUP comes from the limitation of present methods to identify cancer origin, despite an often complicated and costly process which can significantly delay proper treatment of such patients. Recent studies revealed a high degree of variation in clinical management, in the absence of evidence based treatment for CUP¹¹. Many protocols were evaluated¹² but have shown relatively small benefit¹³. Determining tumor tissue of origin is thus an important clinical application of molecular diagnostics⁹.

Molecular classification studies for tumor tissue origin¹⁴⁻¹⁷ have generally used classification algorithms that did not utilize domain-specific knowledge: tissues were treated as a-priori equivalents, ignoring underlying similarities between tissue types with a common developmental origin in embryogenesis. An exception of note is the study by Shedden and co-workers¹⁸, that was based on a pathology classification tree. These studies used machine-learning methods that average effects of biological features (e.g. mRNA expression levels), an approach which is more amenable to automated processing but does not use or generate mechanistic insights.

Various markers have been proposed to indicate specific types of cancers and tumor tissue of origin. However, the diagnostic accuracy of tumor markers has not yet been defined. Therefore, there is a need for a more efficient and effective method for diagnosing and classifying specific types of cancers.

### SUMMARY OF THE INVENTION

The present invention provides specific nucleic acid sequences for use in the identification, classification and diagnosis of specific cancers and tumor tissue of origin. The nucleic acid sequences can also be used as prognostic markers for prognostic evaluation and determination of appropriate treatment of a subject based on the abundance of the nucleic acid sequences in a biological sample.

The invention is based in part on the development of a microRNA-based classifier for tumor classification. microRNA expression levels were measured in 400 paraffin-embedded and fresh-frozen samples from 22 different tumor tissues and metastases. microRNA microarray data of 253 samples was used to construct a classifier, based on 48 microRNAs, each linked to specific differential-diagnosis roles. Two-thirds of the samples were classified with high-confidence, with accuracy exceeding 90%. In an independent blinded test-set of 83 samples, overall high-confidence accuracy reached 89%. Classification accuracy reached 100% for most tissue classes, including 131 metastatic samples. The significance of the microRNA biomarkers was further validated by a sensitive qRT-PCR using 65 additional blinded test samples. The findings demonstrate the utility of microRNA as novel biomarkers for CUP. The classifier produces statistically meaningful confidence measures and may have wide biological as well as diagnostic applications.

According to a first aspect, the present invention provides a method of identifying a tissue of origin of a biological sample, the method comprising: obtaining a biological sample from a subject; determining expression of individual nucleic acids in a predetermined set of microRNAs; and classifying the tissue of origin for said sample by a classifier. According to one embodiment, said classifier is a decision tree model.

According to another aspect, the present invention provides a method of classifying a tissue of origin of a biological sample, the method comprising: obtaining a biological sample from a subject; determining an expression profile in said sample of nucleic acid sequences selected from the group consisting of SEQ ID NOS: 1-96, or a sequence having at least about 80% identity thereto; and comparing said expression profile to a reference expression profile; whereby the differential expression of any of said nucleic acid sequences allows the identification of the tissue of origin of said sample.

According to certain embodiments, said tissue is selected from the group consisting of liver, lung, bladder, prostate, breast, colon, ovary, testis, stomach, thyroid, pancreas, brain, endometrium, head and neck, lymph node, kidney, melanocytes, meninges, thymus, gastrointestinal and prostate.

According to some embodiments said biological sample is a cancerous sample.

According to another aspect, the present invention provides a method of classifying a cancer or hyperplasia, the method comprising: obtaining a biological sample from a subject; measuring the relative abundance in said sample of nucleic acid sequences selected from the group consisting of SEQ ID NOS: 1-96 or a sequence having at least about 80% identity thereto; and comparing said obtained measurement to a reference value representing abundance of said nucleic acid; whereby the differential expression of any of said nucleic acid sequences allows the classification of said cancer or hyperplasia.

According to one embodiment, said sample is obtained from a subject with a metastatic cancer. According to another embodiment, said sample is obtained from a subject with cancer of unknown primary (CUP). According to a further embodiment, said sample is obtained from a subject with a primary cancer. According to still another embodiment, said sample is a tumor of unidentified origin, a metastatic tumor or a primary tumor.

According to certain embodiments, said cancer is selected from the group consisting of liver cancer, lung cancer, bladder cancer, prostate cancer, breast cancer, colon cancer, ovarian cancer, testicular cancer, stomach cancer, thyroid cancer, pancreas cancer, brain cancer, endometrium cancer, head and neck cancer, lymph node cancer, kidney cancer, melanoma, meninges cancer, thymus cancer, prostate cancer, gastrointestinal stromal cancer and sarcoma.

According to some embodiments, said cancer is a lung cancer selected from the group consisting of lung carcinoid, lung pleural mesothelioma and lung squamous cell carcinoma.

According to other embodiments, said biological sample is selected from the group consisting of bodily fluid, a cell line and a tissue sample. According to some embodiments, said tissue is a fresh, frozen, fixed, wax-embedded or formalin fixed paraffin-embedded (FFPE) tissue.

The classification method of the present invention further comprises use of at least one classifier algorithm, said classifier algorithm is selected from the group consisting of decision tree classifier, logistic regression classifier, linear regression classifier, nearest neighbor classifier (including K nearest neighbors), neural network classifier, Gaussian mixture model (GMM) classifier and Support Vector Machine (SVM) classifier. The classifier may use a decision tree structure (including binary tree) or a voting (including weighted voting) scheme to compare the classification of one or more classifier algorithms in order to reach a unified or majority decision.

The invention further provides a method for classifying a cancer of liver origin, the method comprising measuring the relative abundance of a nucleic acid sequence selected from the group consisting of SEQ ID NOS: 1-4, or a sequence having at least about 80% identity thereto in a sample obtained from a subject; wherein the abundance of said nucleic acid sequence is indicative of a cancer of liver origin.

The invention further provides a method for classifying a cancer of testicular origin, the method comprising measuring the relative abundance of a nucleic acid sequence selected from the group consisting of SEQ ID NOS: 1-6, or a sequence having at least about 80% identity thereto in a sample obtained from a subject; wherein the abundance of said nucleic acid sequence is indicative of a cancer of testicular origin.

The invention further provides a method for classifying a cancer of lung origin, the method comprising measuring the relative abundance of a nucleic acid sequence selected from the group consisting of SEQ ID NOS: 1-8, 25, 26, 33, 34, 37, 38, 45, 46, 49, 50, 57-64, 69-84, 95 and 96, or a sequence having at least about 80% identity thereto in a sample obtained from a subject; wherein the abundance of said nucleic acid sequence is indicative of a cancer of lung origin.

The invention further provides a method for classifying a cancer of lung carcinoid origin, the method comprising measuring the relative abundance of a nucleic acid sequence selected from the group consisting of SEQ ID NOS: 1-8, 31, 32, 37, 38, 45-48, 95 and 96, or a sequence having at least about 80% identity thereto in a sample obtained from a subject; wherein the abundance of said nucleic acid sequence is indicative of a cancer of lung carcinoid origin.

The invention further provides a method for classifying a cancer of lung pleura origin, the method comprising measuring the relative abundance of a nucleic acid sequence selected from the group consisting of SEQ ID NOS: 1-14, 19-40, 95 and 96, or a sequence having at least about 80% identity thereto in a sample obtained from a subject; wherein the abundance of said nucleic acid sequence is indicative of a cancer of lung pleura origin.

The invention further provides a method for classifying a cancer of lung squamous origin, the method comprising measuring the relative abundance of a nucleic acid sequence selected from the group consisting of SEQ ID NOS: 1-8, 29, 30, 33, 34, 37, 38, 45, 46, 57-64, 69-74, 85, 86 and 89-96, or a sequence having at least about 80% identity thereto in a sample obtained from a subject; wherein the abundance of said nucleic acid sequence is indicative of a cancer of lung squamous origin.

The invention further provides a method for classifying a cancer of pancreatic origin, the method comprising measuring the relative abundance of a nucleic acid sequence selected from the group consisting of SEQ ID NOS: 1-8, 31, 32, 37, 38, 45-56, 95 and 96, or a sequence having at least about 80% identity thereto in a sample obtained from a subject; wherein the abundance of said nucleic acid sequence is indicative of a cancer of pancreatic origin.

The invention further provides a method for classifying a cancer of brain origin, the method comprising measuring the relative abundance of a nucleic acid sequence selected from the group consisting of SEQ ID NOS: 1-14, 19-24, 95 and 96, or a sequence having at least about 80% identity thereto in a sample obtained from a subject; wherein the abundance of said nucleic acid sequence is indicative of a cancer of brain origin.

The invention further provides a method for classifying a cancer of breast origin, the method comprising measuring the relative abundance of a nucleic acid sequence selected from the group consisting of SEQ ID NOS: 1-8, 33, 34, 37, 38, 45, 46, 49, 50, 57-68, 95 and 96, or a sequence having at least about 80% identity thereto in a sample obtained from a subject; wherein the abundance of said nucleic acid sequence is indicative of a cancer of breast origin.

The invention further provides a method for classifying a cancer of prostate origin, the method comprising measuring the relative abundance of a nucleic acid sequence selected from the group consisting of SEQ ID NOS: 1-8, 33, 34, 37, 38, 45, 46, 49, 50, 57-68, 95 and 96, or a sequence having at least about 80% identity thereto in a sample obtained from a subject; wherein the abundance of said nucleic acid sequence is indicative of a cancer of prostate origin.

The invention further provides a method for classifying a cancer of endometrium origin, the method comprising measuring the relative abundance of a nucleic acid sequence selected from the group consisting of SEQ ID NOS: 1-8, 33, 34, 37, 38, 45, 46, 49, 50, 57-64, 69-90, 95 and 96, or a sequence having at least about 80% identity thereto in a sample obtained from a subject; wherein the abundance of said nucleic acid sequence is indicative of a cancer of endometrium origin.

The invention further provides a method for classifying a cancer of thyroid origin, the method comprising measuring the relative abundance of a nucleic acid sequence selected from the group consisting of SEQ ID NOS: 1-8, 33, 34, 37, 38, 45, 46, 49, 50, 57-64, 69-78, 95 and 96, or a sequence having at least about 80% identity thereto in a sample obtained from a subject; wherein the abundance of said nucleic acid sequence is indicative of a cancer of thyroid origin.

The invention further provides a method for classifying a cancer of head and neck origin, the method comprising measuring the relative abundance of a nucleic acid sequence selected from the group consisting of SEQ ID NOS: 1-8, 29, 30, 33, 34, 37, 38, 45, 46, 57-64, 69-74, 85, 86, and 89-96, or a sequence having at least about 80% identity thereto in a sample obtained from a subject; wherein the abundance of said nucleic acid sequence is indicative of a cancer of head and neck.

The invention further provides a method for classifying a cancer of colon origin, the method comprising measuring the relative abundance of a nucleic acid sequence selected from the group consisting of SEQ ID NOS: 1-8, 31, 32, 37, 38, 45-52, 95 and 96, or a sequence having at least about 80% identity thereto in a sample obtained from a subject; wherein the abundance of said nucleic acid sequence is indicative of a cancer of colon origin.

The invention further provides a method for classifying a cancer of bladder origin, the method comprising measuring the relative abundance of a nucleic acid sequence selected from the group consisting of SEQ ID NOS: 1-8, 25, 26, 33, 34, 37, 38, 45, 46, 49, 50, 57-64, 69-84, 95 and 96, or a sequence having at least about 80% identity thereto in a sample obtained from a subject; wherein the abundance of said nucleic acid sequence is indicative of a cancer of bladder origin.

The invention further provides a method for classifying a cancer of ovarian origin, the method comprising measuring the relative abundance of a nucleic acid sequence selected from the group consisting of SEQ ID NOS: 1-8, 33, 34, 37, 38, 45, 46, 49, 50, 57-64, 69-90, 95 and 96, or a sequence having at least about 80% identity thereto in a sample obtained from a subject; wherein the abundance of said nucleic acid sequence is indicative of a cancer of ovarian origin.

The invention further provides a method for classifying a cancer of lymph node origin, the method comprising measuring the relative abundance of a nucleic acid sequence selected from the group consisting of SEQ ID NOS: 1-18, 95 and 96, or a sequence having at least about 80% identity thereto in a sample obtained from a subject; wherein the abundance of said nucleic acid sequence is indicative of a cancer of lymph node origin.

The invention further provides a method for classifying a cancer of kidney origin, the method comprising measuring the relative abundance of a nucleic acid sequence selected from the group consisting of SEQ ID NOS: 1-14, 19-40, 95 and 96, or a sequence having at least about 80% identity thereto in a sample obtained from a subject; wherein the abundance of said nucleic acid sequence is indicative of a cancer of kidney origin.

The invention further provides a method for classifying a cancer of melanocytes origin, the method comprising measuring the relative abundance of a nucleic acid sequence selected from the group consisting of SEQ ID NOS: 1-18, 95 and 96, or a sequence having at least about 80% identity thereto in a sample obtained from a subject; wherein the abundance of said nucleic acid sequence is indicative of a cancer of melanocytes origin.

The invention further provides a method for classifying a cancer of meninges origin, the method comprising measuring the relative abundance of a nucleic acid sequence selected from the group consisting of SEQ ID NOS: 1-14, 19-28, 95 and 96, or a sequence having at least about 80% identity thereto in a sample obtained from a subject; wherein the abundance of said nucleic acid sequence is indicative of a cancer of meninges origin.

The invention further provides a method for classifying a cancer of thymus (thymoma—type B2) origin, the method comprising measuring the relative abundance of a nucleic acid sequence selected from the group consisting of SEQ ID NOS: 1-14, 19-28, 95 and 96, or a sequence having at least about 80% identity thereto in a sample obtained from a subject; wherein the abundance of said nucleic acid sequence is indicative of a cancer of thymus (thymoma—type B2) origin.

The invention further provides a method for classifying a cancer of thymus (thymoma—type B3) origin, the method comprising measuring the relative abundance of a nucleic acid sequence selected from the group consisting of SEQ ID NOS: 1-8, 29, 30, 33, 34, 37, 38, 45, 46, 49, 50, 57-64, 69-78, 95 and 96, or a sequence having at least about 80% identity thereto in a sample obtained from a subject; wherein the abundance of said nucleic acid sequence is indicative of a cancer of thymus (thymoma—type B3) origin.

The invention further provides a method for classifying a cancer of gastrointestinal stromal origin, the method comprising measuring the relative abundance of a nucleic acid sequence selected from the group consisting of SEQ ID NOS: 1-14, 19-36, 41-44, 95 and 96, or a sequence having at least about 80% identity thereto in a sample obtained from a subject; wherein the abundance of said nucleic acid sequence is indicative of a cancer of.

The invention further provides a method for classifying a cancer of sarcoma origin, the method comprising measuring the relative abundance of a nucleic acid sequence selected from the group consisting of SEQ ID NOS: 1-14, 19-36, 41-44, 95 and 96, or a sequence having at least about 80% identity thereto in a sample obtained from a subject; wherein the abundance of said nucleic acid sequence is indicative of a cancer of gastrointestinal stromal origin.

The invention further provides a method for classifying a cancer of stomach origin, the method comprising measuring the relative abundance of a nucleic acid sequence selected from the group consisting of SEQ ID NOS: 1-8, 31, 32, 37, 38, 45-56, 95 and 96, or a sequence having at least about 80% identity thereto in a sample obtained from a subject; wherein the abundance of said nucleic acid sequence is indicative of a cancer of stomach origin.

According to another aspect, the present invention provides a kit for cancer classification, said kit comprising a probe comprising a nucleic acid sequence selected from the group consisting of SEQ ID NOS: 1-96; a complementary sequence thereof; and sequence having at least about 80% identity thereto.

These and other embodiments of the present invention will become apparent in conjunction with the figures, description and claims that follow.

### BRIEF DESCRIPTION OF THE DRAWINGS

FIG. 1 shows comparison of microRNA expression in primary and metastatic tumor samples. A) Primary and metastatic colon cancer samples are compared, and p-values (unpaired t-test on the log-signal) are calculated for each microRNA that passes a signal threshold in at least one of the sets. The sorted p-values agree with a random distribution of p-values (uniform in the range 0-1, dotted black line). The lower line indicates the 10% false discovery rate (FDR) line—p-values below this line have a 10% probability of false discovery. For colon cancer metastases, none of the features passes a 10% false-discovery test. B) Dot-plot of the mean log₂ signals of the primary vs. the metastatic colon cancer samples (crosses; dotted line is a guide to the eye and shows the diagonal where mean expression is equal). C) Comparison (as in A) of primary stomach cancers to stomach cancer metastases to the lymph nodes. The first three microRNAs with lowest p-values pass the false discovery test (at 10% false discovery rate). D) Dot-plot (as in B) of the primary stomach cancers vs. stomach metastases to the lymph node. The three microRNAs that pass the FDR test are highlighted: miR-133a (SEQ ID NO: 97) and miR-143 (SEQ ID NO: 99) are over-expressed in the primary tumors, miR-150 (SEQ ID NO: 101) is over-expressed in the metastases.

FIG. 2 demonstrates the structure of the decision-tree classifier, with 24 nodes (numbered, Table 2) and 25 leaves. Each node is a binary decision between two sets of samples, those to the left and right of the node. A series of binary decisions, starting at node #1 and moving downwards, lead to one of the possible tumor types, which are the “leaves” of the tree. A sample which is classified to the left branch at node #1 is assigned to the “liver” class, otherwise it continues to node #2. Decisions are made at consecutive nodes using microRNA expression levels, until an end-point (“leaf” of the tree) is reached, indicating the predicted class for this sample. For example, a sample which is classified as “breast” must undergo the path through nodes #1, #2, #3, #12, #16, and #17, taking the left branch at nodes #3, #16 and #17 and the right branch at nodes #1, #2 and #12, and no decision is needed at any of the other nodes. In specifying the tree structure, we combined clinico-pathological considerations with properties observed in the training set data. For example, thymus samples separated into two groups according to their histological types, differing in the expression of epithelial-related microRNAs, ostensibly due to the higher proportion of lymphocytes in B2-type tumors. The first major division (node #3) separates tissues of epithelial origin from tissues of other or mixed origin, a biological difference which is reflected in their microRNA expression profiles, especially in expression of the miR-141 (SEQ ID NO: 69)/200 (SEQ ID NOs: 3, 11) family. Thymus B2 tumors are here grouped with non-epithelial or mixed tissues (on the right branch), and are separated from these later (FIG. 4). Liver and testis were placed first in the tree because these tissues contain highly specific expression of microRNAs (hsa-miR-122a (SEQ ID NO: 1) and hsa-miR-372 (SEQ ID NO: 5) respectively) that can be used to easily identify them, reducing interference later. Subsequent nodes recapitulated the separation of the gastrointestinal tract from other epithelial tissues (node #12) using miR-194 (SEQ ID NO: 37) and additional microRNAs (FIG. 3B). Lung carcinoid tumors, as opposed to other types of lung tumors, were found to have high expression of miR-194, which may be related to their distinct biological characteristics. These tumors are therefore grouped with the gastrointestinal tissues at node #12, and separated from them at node #13 using other microRNAs (FIG. 3A). Cancers of the esophagus differed substantially in the expression of microRNAs used for classification according to their histological types: gastroesophageal junction adenocarcinomas were similar to samples of stomach cancer, whereas squamous samples had a strong similarity to the highly squamous head and neck cancers. Thus, the “stomach*” class includes both stomach cancers and gastroesophageal junction adenocarcinomas; the “head and neck*” class includes cancers of head and neck and squamous carcinoma of esophagus. “GIST” indicates gastrointestinal stromal tumors. Additional information such as patient gender or available clinical-pathological information is easy to incorporate into the tree by trimming leaves or branches, without need for retraining.

FIG. 3 demonstrates binary decisions at nodes of the decision-tree. A) When training a decision algorithm for a given node, only those sample classes which are possible outcomes (“leaves”) of this node are used for training. At node #13 (see FIG. 2), lung-carcinoid tumors (triangles, 7 samples) are easily separated from tumors of gastrointestinal origin (grey and empty squares, 49 samples) using the expression levels of hsa-miR-21(SEQ ID NO: 31) and hsa-let-7e (SEQ ID NO: 47) (with one outlier). Other samples which branch out earlier in the tree and are not well-separated by these microRNAs (circles, 283 samples) are not considered. Importantly, metastatic samples of gastrointestinal origin (empty squares, 23 samples) are distributed with the primary tumors. The solid line indicates the values of hsa-miR-21 and hsa-let-7e for which the logistic regression model of node #13 assigns a probability P=0.5. Points above the line are assigned a probability P>0.5 and take the left branch (to node #14), points below the line take the right branch and are classified as lung-carcinoid. B) Expression levels of hsa-miR-194 (SEQ ID NO: 37), hsa-miR-145 (SEQ ID NO: 45), and hsa-miR-205 (SEQ ID NO: 7) at node #12 in the tree (FIG. 2). These microRNAs can be used to separate between the left branch of node #12 (grey squares, 56 samples, empty squares show metastatic samples), i.e. samples from the stomach, pancreas, colon or lung-carcinoid, and other epithelial samples in the right branch of node #12 (grey triangles, 152 samples, empty triangles show metastatic samples). C) Validation of the microRNAs used in node #1 (Table 2) by qRT-PCR: liver (squares, 9 samples) and non-liver samples (triangles, 71 samples) are easily separated using hsa-miR-122a (SEQ ID NO: 1) and has-miR-141 (SEQ ID NO: 69) (FIG. 5). The signal shown for each sample is the difference in cycle threshold (Cₜ) between U6 and the microRNA. A higher difference means higher expression of this microRNA. Liver tumors have higher expression of hsa-miR-122a and lower expression of hsa-miR-141. Line indicates the decision threshold of the logistic regression (FIG. 5). D) Validation of the microRNAs used in node #12 (Table 2) by qRT-PCR: samples of gastrointestinal tumors (squares, 13 samples) show distinct expression levels (FIG. 5) of hsa-miR-145 (SEQ ID NO: 45), hsa-miR-194 (SEQ ID NO: 37), and hsa-miR-205 (SEQ ID NO: 7) compared to other epithelial tumors (triangles, 52 samples). The results obtained by qRT-PCR are very similar to those obtained by the microarray platform at this node (panel B) and show similar distributions.

FIG. 4 demonstrates a logistic regression model in one dimension. The logistic regression model for node #8 in the tree (Table 2) assigns each sample a probability (P, solid curve) of belonging to the group in the left branch (i.e. thymus B2) as a function (inset) of the expression level of hsa-miR-205 (SEQ ID NO: 7) in the sample (M is the natural log of the measured expression level). Bars show the distribution of the expression levels of hsa-miR-205 in thymus B2 samples (left in node #8) and samples (right in node #8). Numbers indicate the number of samples in each bin. Samples with M>9.2 have P>0.5 (dotted grey lines) and are assigned to the thymus class, whereas all other samples are assigned to the right branch at node #8 and continue with classification by other decision nodes.

FIG. 5 demonstrates the accuracy of classification with the qRT-PCR data. The receiver operating characteristic curve (ROC curve) plots the sensitivity against the false-positive rate (one minus the specificity) for different cutoff values of a diagnostic metric, and is a measure of classification performance. The area under the ROC curve (AUC) can be used to assess the diagnostic performance of the metric. A random classifier has AUC=0.5, and an optimal classifier with perfect sensitivity and specificity of 100% has AUC=1.

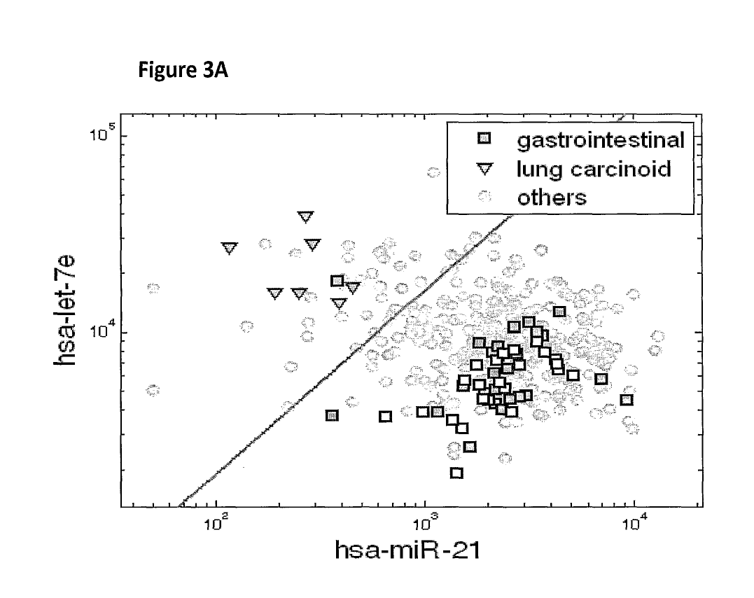

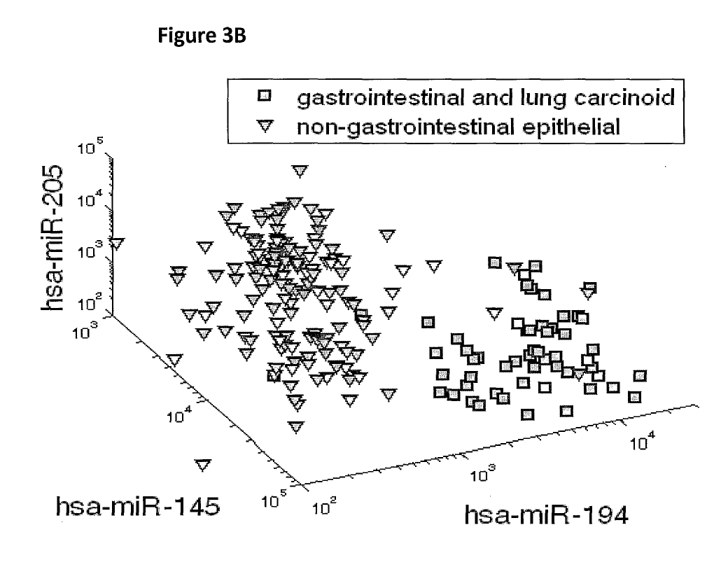

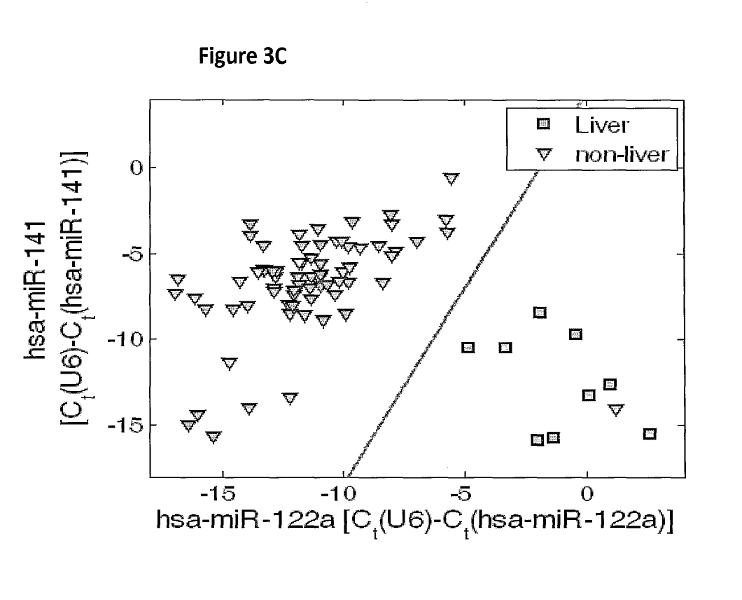

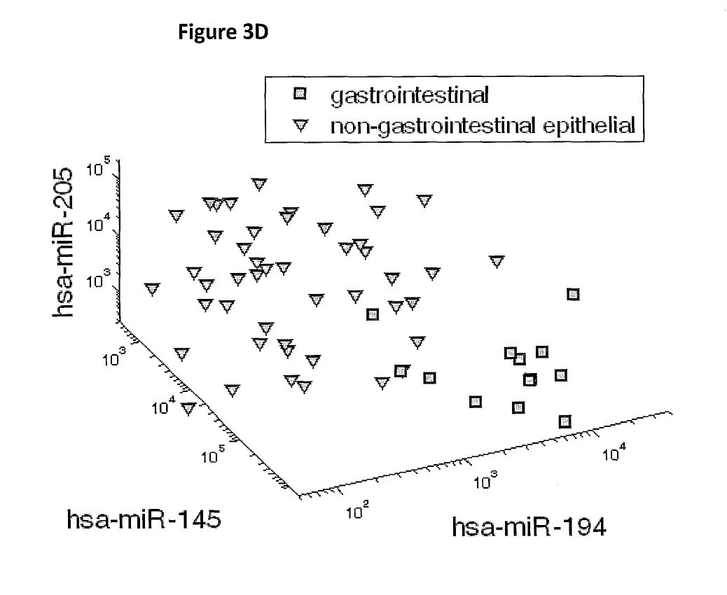

A) Probability (P) output of a logistic classifier trained to separate liver from non-liver samples using the expression levels of hsa-miR-122a (SEQ ID NO: 1) and hsa-miR-141 (SEQ ID NO: 69) measured in qRT-PCR (FIG. 3C). Squares show the 9 liver samples, triangles show the 71 non-liver samples. A threshold at Pₜₕ=0.8 easily separates the two classes, with one outlier.

B) The corresponding ROC curve has AUC=0.988, near the optimum. A circle shows Pₜₕ=0.8 which has 100% sensitivity and 99% specificity in identifying liver samples.

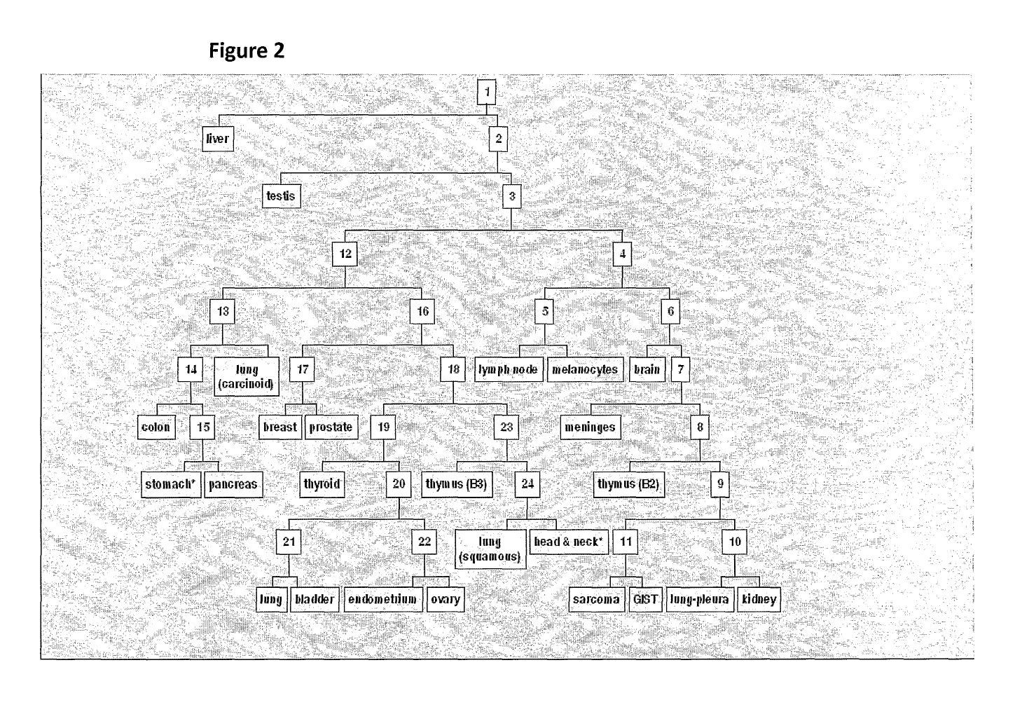

C) Probability (P) output of a logistic classifier trained to separate gastrointestinal (GI) samples from non-GI samples using the expression levels of hsa-miR-145 (SEQ ID NO: 45), hsa-miR194 (SEQ ID NO: 37) and hsa-miR-205 (SEQ ID NO: 7) (at node #12 in the decision-tree, FIG. 2) measured in qRT-PCR (FIG. 3D). Squares show the 13 colon or pancreas samples, triangles show the 52 other epithelial samples (right branch at node #12). A threshold at Pₜₕ=0.5 has 6 errors.

D) The corresponding ROC curve has AUC=0.914. A circle shows Pₜₕ=0.5, which has 92% sensitivity and 91% specificity in identifying the gastrointestinal samples.

### DETAILED DESCRIPTION OF THE INVENTION

The invention is based on the discovery that specific nucleic acid sequences can be used for the classification of cancers. The present invention provides a sensitive, specific and accurate method which can be used to distinguish between different tissues and tumor origins A new microRNA-based classifier was developed for determining tissue origin of tumors that reaches an accuracy of about 90% based on a surprisingly small number of microRNAs. The classifier uses a transparent algorithm and allows a clear interpretation of the specific biomarkers. The classifier uses only 48 microRNA markers to reach an overall accuracy of about 90% among 22 classes, on blinded test samples and on more than 130 metastases. According to the present invention each node in the classification tree may be used as an independent differential diagnosis tool, for example in the identification of different types of lung cancer. The performance of the classifier using a surprisingly small number of markers highlights the utility of microRNA as tissue-specific cancer biomarkers, and provides an effective means for facilitating diagnosis of CUP.

The possibility to distinguish between different tumor origins facilitates providing the patient with the best and most suitable treatment.

The present invention provides diagnostic assays and methods, both quantitative and qualitative for detecting, diagnosing, monitoring, staging and prognosticating cancers by comparing levels of the specific microRNA molecules of the invention. Such levels are preferably measured in at least one of biopsies, tumor samples, cells, tissues and/or bodily fluids. The present invention provides methods for diagnosing the presence of a specific cancer by analyzing changes in levels of said microRNA molecules in biopsies, tumor samples, cells, tissues or bodily fluids.

In the present invention, determining the presence of said microRNA levels in biopsies, tumor samples, cells, tissues or bodily fluid, is particularly useful for discriminating between different cancers.

All the methods of the present invention may optionally further include measuring levels of other cancer markers. Other cancer markers, in addition to said microRNA molecules, useful in the present invention will depend on the cancer being tested and are known to those of skill in the art.

Assay techniques that can be used to determine levels of gene expression, such as the nucleic acid sequence of the present invention, in a sample derived from a patient are well known to those of skill in the art. Such assay methods include, but are not limited to, radioimmunoassays, reverse transcriptase PCR (RT-PCR) assays, immunohistochemistry assays, in situ hybridization assays, competitive-binding assays, Northern Blot analyses, ELISA assays, nucleic acid microarrays and biochip analysis.

In some embodiments of the invention, correlations and/or hierarchical clustering can be used to assess the similarity of the expression level of the nucleic acid sequences of the invention between a specific sample and different exemplars of cancer samples. An arbitrary threshold on the expression level of one or more nucleic acid sequences can be set for assigning a sample or cancer sample to one of two groups. Alternatively, in a preferred embodiment, expression levels of one or more nucleic acid sequences of the invention are combined by a method such as logistic regression to define a metric which is then compared to previously measured samples or to a threshold. The threshold for assignment is treated as a parameter, which can be used to quantify the confidence with which samples are assigned to each class. The threshold for assignment can be scaled to favor sensitivity or specificity, depending on the clinical scenario. The correlation value to the reference data generates a continuous score that can be scaled and provides diagnostic information on the likelihood that a samples belongs to a certain class of cancer origin or type. In multivariate analysis, the microRNA signature provides a high level of prognostic information.

### DEFINITIONS

It is to be understood that the terminology used herein is for the purpose of describing particular embodiments only and is not intended to be limiting. It must be noted that, as used in the specification and the appended claims, the singular forms “a,” “an” and “the” include plural referents unless the context clearly dictates otherwise.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9 and 7.0 are explicitly contemplated.

Aberrant Proliferation

As used herein, the term “aberrant proliferation” means cell proliferation that deviates from the normal, proper, or expected course. For example, aberrant cell proliferation may include inappropriate proliferation of cells whose DNA or other cellular components have become damaged or defective. Aberrant cell proliferation may include cell proliferation whose characteristics are associated with an indication caused by, mediated by, or resulting in inappropriately high levels of cell division, inappropriately low levels of apoptosis, or both. Such indications may be characterized, for example, by single or multiple local abnormal proliferations of cells, groups of cells, or tissue(s), whether cancerous or non-cancerous, benign or malignant.

About

As used herein, the term “about” refers to +/−10%.

Attached

“Attached” or “immobilized” as used herein to refer to a probe and a solid support means that the binding between the probe and the solid support is sufficient to be stable under conditions of binding, washing, analysis, and removal. The binding may be covalent or non-covalent. Covalent bonds may be formed directly between the probe and the solid support or may be formed by a cross linker or by inclusion of a specific reactive group on either the solid support or the probe or both molecules. Non-covalent binding may be one or more of electrostatic, hydrophilic, and hydrophobic interactions. Included in non-covalent binding is the covalent attachment of a molecule, such as streptavidin, to the support and the non-covalent binding of a biotinylated probe to the streptavidin. Immobilization may also involve a combination of covalent and non-covalent interactions.

Biological Sample

“Biological sample” as used herein means a sample of biological tissue or fluid that comprises nucleic acids. Such samples include, but are not limited to, tissue or fluid isolated from subjects. Biological samples may also include sections of tissues such as biopsy and autopsy samples, FFPE samples, frozen sections taken for histological purposes, blood, blood fraction, plasma, serum, sputum, stool, tears, mucus, hair, skin, urine, effusions, ascitic fluid, amniotic fluid, saliva, cerebrospinal fluid, cervical secretions, vaginal secretions, endometrial secretions, gastrointestinal secretions, bronchial secretions, cell line, tissue sample, or secretions from the breast. A biological sample may be provided by removing a sample of cells from a subject but can also be accomplished by using previously isolated cells (e.g., isolated by another person, at another time, and/or for another purpose), or by performing the methods described herein in vivo. Archival tissues, such as those having treatment or outcome history, may also be used. Biological samples also include explants and primary and/or transformed cell cultures derived from animal or human tissues.

Cancer

The term “cancer” is meant to include all types of cancerous growths or oncogenic processes, metastatic tissues or malignantly transformed cells, tissues, or organs, irrespective of histopathologic type or stage of invasiveness. Examples of cancers include but are not limited to solid tumors and leukemias, including: apudoma, choristoma, branchioma, malignant carcinoid syndrome, carcinoid heart disease, carcinoma (e.g., Walker, basal cell, basosquamous, Brown-Pearce, ductal, Ehrlich tumor, non-small cell lung (e.g., lung squamous cell carcinoma, lung adenocarcinoma and lung undifferentiated large cell carcinoma), oat cell, papillary, bronchiolar, bronchogenic, squamous cell, and transitional cell), histiocytic disorders, leukemia (e.g., B cell, mixed cell, null cell, T cell, T-cell chronic, HTLV-1′-associated, lymphocytic acute, lymphocytic chronic, mast cell, and myeloid), histiocytosis malignant, Hodgkin disease, immunoproliferative small, non-Hodgkin lymphoma, plasmacytoma, reticuloendotheliosis, melanoma; chondroblastoma, chondroma, chondrosarcoma, fibroma, fibrosarcoma, giant cell tumors, histiocytoma, lipoma, liposarcoma, mesothelioma, myxoma, myxosarcoma, osteoma, osteosarcoma, Ewing sarcoma, synovioma, adenofibroma, adenolymphoma, carcinosarcoma, chordoma, craniopharyngioma, dysgerminoma, hamartoma, mesenchymoma, mesonephroma, myosarcoma, ameloblastoma, cementoma, odontoma, teratoma, thymoma, trophoblastic tumor, adeno-carcinoma, adenoma, cholangioma, cholesteatoma, cylindroma, cystadenocarcinoma, cystadenoma, granulosa cell tumor, gynandroblastoma, hepatoma, hidradenoma, islet cell tumor, Leydig cell tumor, papilloma, Sertoli cell tumor, theca cell tumor, leiomyoma, leiomyosarcoma, myoblastoma, myosarcoma, rhabdomyoma, rhabdomyosarcoma, ependymoma, ganglioneuroma, glioma, medulloblastoma, meningioma, neurilemmoma, neuroblastoma, neuroepithelioma, neurofibroma, neuroma, paraganglioma, paraganglioma nonchromaffin, angiokeratoma, angiolymphoid hyperplasia with eosinophilia, angioma sclerosing, angiomatosis, glomangioma, hemangioendothelioma, hemangioma, hemangiopericytoma, hemangiosarcoma, lymphangioma, lymphangiomyoma, lymphangiosarcoma, pinealoma, carcinosarcoma, chondrosarcoma, cystosarcoma, phyllodes, fibrosarcoma, hemangiosarcoma, leimyosarcoma, leukosarcoma, liposarcoma, lymphangiosarcoma, myosarcoma, myxosarcoma, ovarian carcinoma, rhabdomyosarcoma, sarcoma (e.g., Ewing, experimental, Kaposi, and mast cell), neurofibromatosis, and cervical dysplasia, and other conditions in which cells have become immortalized or transformed.

Classification

The term classification refers to a procedure and/or algorithm in which individual items are placed into groups or classes based on quantitative information on one or more characteristics inherent in the items (referred to as traits, variables, characters, features, etc) and based on a statistical model and/or a training set of previously labeled items. A “classification tree” is a decision tree that places categorical variables into classes.

Complement

“Complement” or “complementary” as used herein to refer to a nucleic acid may mean Watson-Crick (e.g., A-T/U and C-G) or Hoogsteen base pairing between nucleotides or nucleotide analogs of nucleic acid molecules. A full complement or fully complementary means 100% complementary base pairing between nucleotides or nucleotide analogs of nucleic acid molecules.

Ct

“Ct” as used herein refers to Cycle Threshold of qRT-PCR, which is the fractional cycle number at which the fluorescence crosses the threshold.

Data Processing Routine

As used herein, a “data processing routine” refers to a process that can be embodied in software that determines the biological significance of acquired data (i.e., the ultimate results of an assay or analysis). For example, the data processing routine can make determination of tissue of origin based upon the data collected. In the systems and methods herein, the data processing routine can also control the data collection routine based upon the results determined. The data processing routine and the data collection routines can be integrated and provide feedback to operate the data acquisition, and hence provide assay-based judging methods.

Data Set

As use herein, the term “data set” refers to numerical values obtained from the analysis. These numerical values associated with analysis may be values such as peak height and area under the curve.

Data Structure

As used herein the term “data structure” refers to a combination of two or more data sets, applying one or more mathematical manipulations to one or more data sets to obtain one or more new data sets, or manipulating two or more data sets into a form that provides a visual illustration of the data in a new way. An example of a data structure prepared from manipulation of two or more data sets would be a hierarchical cluster.

Detection

“Detection” means detecting the presence of a component in a sample. Detection also means detecting the absence of a component. Detection also means determining the level of a component, either quantitatively or qualitatively.

Differential Expression

“Differential expression” means qualitative or quantitative differences in the temporal and/or spatial gene expression patterns within and among cells and tissue. Thus, a differentially expressed gene may qualitatively have its expression altered, including an activation or inactivation, in, e.g., normal versus diseased tissue. Genes may be turned on or turned off in a particular state, relative to another state thus permitting comparison of two or more states. A qualitatively regulated gene may exhibit an expression pattern within a state or cell type which may be detectable by standard techniques. Some genes may be expressed in one state or cell type, but not in both. Alternatively, the difference in expression may be quantitative, e.g., in that expression is modulated, up-regulated, resulting in an increased amount of transcript, or down-regulated, resulting in a decreased amount of transcript. The degree to which expression differs needs only be large enough to quantify via standard characterization techniques such as expression arrays, quantitative reverse transcriptase PCR, Northern blot analysis, real-time PCR, in situ hybridization and RNase protection.

Expression Profile

The term “expression profile” is used broadly to include a genomic expression profile, e.g., an expression profile of microRNAs. Profiles may be generated by any convenient means for determining a level of a nucleic acid sequence e.g. quantitative hybridization of microRNA, labeled microRNA, amplified microRNA, cDNA, etc., quantitative PCR, ELISA for quantitation, and the like, and allow the analysis of differential gene expression between two samples. A subject or patient tumor sample, e.g., cells or collections thereof, e.g., tissues, is assayed. Samples are collected by any convenient method, as known in the art. Nucleic acid sequences of interest are nucleic acid sequences that are found to be predictive, including the nucleic acid sequences provided above, where the expression profile may include expression data for 5, 10, 20, 25, 50, 100 or more of, including all of the listed nucleic acid sequences. According to some embodiments, the term “expression profile” means measuring the abundance of the nucleic acid sequences in the measured samples.

Expression Ratio

“Expression ratio” as used herein refers to relative expression levels of two or more nucleic acids as determined by detecting the relative expression levels of the corresponding nucleic acids in a biological sample.

Gene

“Gene” as used herein may be a natural (e.g., genomic) or synthetic gene comprising transcriptional and/or translational regulatory sequences and/or a coding region and/or non-translated sequences (e.g., introns, 5′- and 3′-untranslated sequences). The coding region of a gene may be a nucleotide sequence coding for an amino acid sequence or a functional RNA, such as tRNA, rRNA, catalytic RNA, siRNA, miRNA or antisense RNA. A gene may also be an mRNA or cDNA corresponding to the coding regions (e.g., exons and miRNA) optionally comprising 5′- or 3′-untranslated sequences linked thereto. A gene may also be an amplified nucleic acid molecule produced in vitro comprising all or a part of the coding region and/or 5′- or 3′-untranslated sequences linked thereto.

Groove Binder/Minor Groove Binder (MGB)

“Groove binder” and/or “minor groove binder” may be used interchangeably and refer to small molecules that fit into the minor groove of double-stranded DNA, typically in a sequence-specific manner. Minor groove binders may be long, flat molecules that can adopt a crescent-like shape and thus, fit snugly into the minor groove of a double helix, often displacing water. Minor groove binding molecules may typically comprise several aromatic rings connected by bonds with torsional freedom such as furan, benzene, or pyrrole rings. Minor groove binders may be antibiotics such as netropsin, distamycin, berenil, pentamidine and other aromatic diamidines, Hoechst 33258, SN 6999, aureolic anti-tumor drugs such as chromomycin and mithramycin, CC-1065, dihydrocyclopyrroloindole tripeptide (DPI₃), 1,2-dihydro-(3H)-pyrrolo[3,2-e]indole-7-carboxylate (CDPI₃), and related compounds and analogues, including those described in Nucleic Acids in Chemistry and Biology, 2d ed., Blackburn and Gait, eds., Oxford University Press, 1996, and PCT Published Application No. WO 03/078450, the contents of which are incorporated herein by reference. A minor groove binder may be a component of a primer, a probe, a hybridization tag complement, or combinations thereof. Minor groove binders may increase the Tₘ of the primer or a probe to which they are attached, allowing such primers or probes to effectively hybridize at higher temperatures.

Host Cell

“Host cell” as used herein may be a naturally occurring cell or a transformed cell that may contain a vector and may support replication of the vector. Host cells may be cultured cells, explants, cells in vivo, and the like. Host cells may be prokaryotic cells such as E. coli, or eukaryotic cells such as yeast, insect, amphibian, or mammalian cells, such as CHO and HeLa cells.

Identity

“Identical” or “identity” as used herein in the context of two or more nucleic acids or polypeptide sequences mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA sequences, thymine (T) and uracil (U) may be considered equivalent. Identity may be performed manually or by using a computer sequence algorithm such as BLAST or BLAST 2.0.

In Situ Detection

“In situ detection” as used herein means the detection of expression or expression levels in the original site hereby meaning in a tissue sample such as biopsy.

K-Nearest Neighbor

The phrase “k-nearest neighbor” refers to a classification method that classifies a point by calculating the distances between the point and points in the training data set. Then it assigns the point to the class that is most common among its k-nearest neighbors (where k is an integer).

Label

“Label” as used herein means a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and other entities which can be made detectable. A label may be incorporated into nucleic acids and proteins at any position.

Node

A “node” is a decision point in a classification (i.e., decision) tree. Also, a point in a neural net that combines input from other nodes and produces an output through application of an activation function. A “leaf” is a node not further split, the terminal grouping in a classification or decision tree.

Nucleic Acid

“Nucleic acid” or “oligonucleotide” or “polynucleotide”, as used herein means at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequences. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

A nucleic acid will generally contain phosphodiester bonds, although nucleic acid analogs may be included that may have at least one different linkage, e.g., phosphoramidate, phosphorothioate, phosphorodithioate, or O-methylphophoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated herein by reference. Nucleic acids containing one or more non-naturally occurring or modified nucleotides are also included within one definition of nucleic acids. The modified nucleotide analog may be located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino) propyl uridine, 5-bromo uridine; adenosines and guanosines modified at the 8-position, e.g. 8-bromo guanosine; deaza nucleotides, e.g. 7-deaza-adenosine; O- and N-alkylated nucleotides, e.g. N6-methyl adenosine are suitable. The 2′-OH-group may be replaced by a group selected from H, OR, R, halo, SH, SR, NH2, NHR, NR2 or CN, wherein R is C1-C6 alkyl, alkenyl or alkynyl and halo is F, Cl, Br or I. Modified nucleotides also include nucleotides conjugated with cholesterol through, e.g., a hydroxyprolinol linkage as described in Krutzfeldt et al., Nature 438:685-689 (2005), Soutschek et al., Nature 432:173-178 (2004), and U.S. Patent Publication No. 20050107325, which are incorporated herein by reference. Additional modified nucleotides and nucleic acids are described in U.S. Patent Publication No. 20050182005, which is incorporated herein by reference. Modifications of the ribose-phosphate backbone may be done for a variety of reasons, e.g., to increase the stability and half-life of such molecules in physiological environments, to enhance diffusion across cell membranes, or as probes on a biochip. The backbone modification may also enhance resistance to degradation, such as in the harsh endocytic environment of cells. The backbone modification may also reduce nucleic acid clearance by hepatocytes, such as in the liver and kidney. Mixtures of naturally occurring nucleic acids and analogs may be made; alternatively, mixtures of different nucleic acid analogs, and mixtures of naturally occurring nucleic acids and analogs may be made.

Probe

“Probe” as used herein means an oligonucleotide capable of binding to a target nucleic acid of complementary sequence through one or more types of chemical bonds, usually through complementary base pairing, usually through hydrogen bond formation. Probes may bind target sequences lacking complete complementarity with the probe sequence depending upon the stringency of the hybridization conditions. There may be any number of base pair mismatches which will interfere with hybridization between the target sequence and the single stranded nucleic acids described herein. However, if the number of mutations is so great that no hybridization can occur under even the least stringent of hybridization conditions, the sequence is not a complementary target sequence. A probe may be single stranded or partially single and partially double stranded. The strandedness of the probe is dictated by the structure, composition, and properties of the target sequence. Probes may be directly labeled or indirectly labeled such as with biotin to which a streptavidin complex may later bind.

Reference Value

As used herein the term “reference value” means a value that statistically correlates to a particular outcome when compared to an assay result. In preferred embodiments the reference value is determined from statistical analysis of studies that compare microRNA expression with known clinical outcomes.

Stringent Hybridization Conditions

“Stringent hybridization conditions” as used herein mean conditions under which a first nucleic acid sequence (e.g., probe) will hybridize to a second nucleic acid sequence (e.g., target), such as in a complex mixture of nucleic acids. Stringent conditions are sequence-dependent and will be different in different circumstances. Stringent conditions may be selected to be about 5-10° C. lower than the thermal melting point (Tₘ) for the specific sequence at a defined ionic strength pH. The Tₘ may be the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

Substantially Complementary

“Substantially complementary” as used herein means that a first sequence is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides, or that the two sequences hybridize under stringent hybridization conditions.

Substantially Identical

“Substantially identical” as used herein means that a first and a second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or with respect to nucleic acids, if the first sequence is substantially complementary to the complement of the second sequence.

Subject

As used herein, the term “subject” refers to a mammal, including both human and other mammals. The methods of the present invention are preferably applied to human subjects.

Target Nucleic Acid

“Target nucleic acid” as used herein means a nucleic acid or variant thereof that may be bound by another nucleic acid. A target nucleic acid may be a DNA sequence. The target nucleic acid may be RNA. The target nucleic acid may comprise a mRNA, tRNA, shRNA, siRNA or Piwi-interacting RNA, or a pri-miRNA, pre-miRNA, miRNA, or anti-miRNA.

The target nucleic acid may comprise a target miRNA binding site or a variant thereof. One or more probes may bind the target nucleic acid. The target binding site may comprise 5-100 or 10-60 nucleotides. The target binding site may comprise a total of 5, 6, 7, 8, 9, 10, 11, 12, 13, 14, 15, 16, 17, 18, 19, 20, 21, 22, 23, 24, 25, 26, 27, 28, 29, 30-40, 40-50, 50-60, 61, 62 or 63 nucleotides. The target site sequence may comprise at least 5 nucleotides of the sequence of a target miRNA binding site disclosed in U.S. patent application Ser. Nos. 11/384,049, 11/418,870 or 11/429,720, the contents of which are incorporated herein.

Tissue Sample

As used herein, a tissue sample is tissue obtained from a tissue biopsy using methods well known to those of ordinary skill in the related medical arts. The phrase “suspected of being cancerous” as used herein means a cancer tissue sample believed by one of ordinary skill in the medical arts to contain cancerous cells. Methods for obtaining the sample from the biopsy include gross apportioning of a mass, microdissection, laser-based microdissection, or other art-known cell-separation methods.

Tumor

“Tumor” as used herein, refers to all neoplastic cell growth and proliferation, whether malignant or benign, and all pre-cancerous and cancerous cells and tissues.

Variant

“Variant” as used herein referring to a nucleic acid means (i) a portion of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequence substantially identical thereto.

Wild Type

As used herein, the term “wild type” sequence refers to a coding, a non-coding or an interface sequence which is an allelic form of sequence that performs the natural or normal function for that sequence. Wild type sequences include multiple allelic forms of a cognate sequence, for example, multiple alleles of a wild type sequence may encode silent or conservative changes to the protein sequence that a coding sequence encodes.

The present invention employs miRNAs for the identification, classification and diagnosis of specific cancers and the identification of their tissues of origin.

microRNA processing

A gene coding for microRNA (miRNA) may be transcribed leading to production of a miRNA primary transcript known as the pri-miRNA. The pri-miRNA may comprise a hairpin with a stem and loop structure. The stem of the hairpin may comprise mismatched bases. The pri-miRNA may comprise several hairpins in a polycistronic structure.

The hairpin structure of the pri-miRNA may be recognized by Drosha, which is an RNase III endonuclease. Drosha may recognize terminal loops in the pri-miRNA and cleave approximately two helical turns into the stem to produce a 60-70 nt precursor known as the pre-miRNA. Drosha may cleave the pri-miRNA with a staggered cut typical of RNase III endonucleases yielding a pre-miRNA stem loop with a 5′ phosphate and ˜2 nucleotide 3′ overhang. Approximately one helical turn of stem (˜10 nucleotides) extending beyond the Drosha cleavage site may be essential for efficient processing. The pre-miRNA may then be actively transported from the nucleus to the cytoplasm by Ran-GTP and the export receptor Ex-portin-5.

The pre-miRNA may be recognized by Dicer, which is also an RNase III endonuclease. Dicer may recognize the double-stranded stem of the pre-miRNA. Dicer may also off the terminal loop two helical turns away from the base of the stem loop leaving an additional 5′ phosphate and ˜2 nucleotide 3′ overhang. The resulting siRNA-like duplex, which may comprise mismatches, comprises the mature miRNA and a similar-sized fragment known as the miRNA*. The miRNA and miRNA* may be derived from opposing arms of the pri-miRNA and pre-miRNA. MiRNA* sequences may be found in libraries of cloned miRNAs but typically at lower frequency than the miRNAs.

Although initially present as a double-stranded species with miRNA*, the miRNA may eventually become incorporated as a single-stranded RNA into a ribonucleoprotein complex known as the RNA-induced silencing complex (RISC). Various proteins can form the RISC, which can lead to variability in specificity for miRNA/miRNA* duplexes, binding site of the target gene, activity of miRNA (repress or activate), and which strand of the miRNA/miRNA* duplex is loaded in to the RISC.

When the miRNA strand of the miRNA:miRNA* duplex is loaded into the RISC, the miRNA* may be removed and degraded. The strand of the miRNA:miRNA* duplex that is loaded into the RISC may be the strand whose 5′ end is less tightly paired. In cases where both ends of the miRNA:miRNA* have roughly equivalent 5′ pairing, both miRNA and miRNA* may have gene silencing activity.

The RISC may identify target nucleic acids based on high levels of complementarity between the miRNA and the mRNA, especially by nucleotides 2-7 of the miRNA. Only one case has been reported in animals where the interaction between the miRNA and its target was along the entire length of the miRNA. This was shown for mir-196 and Hox B8 and it was further shown that mir-196 mediates the cleavage of the Hox B8 mRNA (Yekta et al 2004, Science 304-594). Otherwise, such interactions are known only in plants (Bartel & Bartel 2003, Plant Physiol 132-709).

A number of studies have looked at the base-pairing requirement between miRNA and its mRNA target for achieving efficient inhibition of translation (reviewed by Bartel 2004, Cell 116-281). In mammalian cells, the first 8 nucleotides of the miRNA may be important (Doench & Sharp 2004 GenesDev 2004-504). However, other parts of the microRNA may also participate in mRNA binding. Moreover, sufficient base pairing at the 3′ can compensate for insufficient pairing at the 5′ (Brennecke et al, 2005 PLoS 3-e85). Computation studies, analyzing miRNA binding on whole genomes have suggested a specific role for bases 2-7 at the 5′ of the miRNA in target binding but the role of the first nucleotide, found usually to be “A” was also recognized (Lewis et al, 2005 Cell 120-15). Similarly, nucleotides 1-7 or 2-8 were used to identify and validate targets by Krek et al (2005, Nat Genet 37-495).

The target sites in the mRNA may be in the 5′ UTR, the 3′ UTR or in the coding region. Interestingly, multiple miRNAs may regulate the same mRNA target by recognizing the same or multiple sites. The presence of multiple miRNA binding sites in most genetically identified targets may indicate that the cooperative action of multiple RISCs provides the most efficient translational inhibition.

miRNAs may direct the RISC to downregulate gene expression by either of two mechanisms: mRNA cleavage or translational repression. The miRNA may specify cleavage of the mRNA if the mRNA has a certain degree of complementarity to the miRNA. When a miRNA guides cleavage, the cut may be between the nucleotides pairing to residues 10 and 11 of the miRNA. Alternatively, the miRNA may repress translation if the miRNA does not have the requisite degree of complementarity to the miRNA. Translational repression may be more prevalent in animals since animals may have a lower degree of complementarity between the miRNA and binding site.

It should be noted that there may be variability in the 5′ and 3′ ends of any pair of miRNA and miRNA*. This variability may be due to variability in the enzymatic processing of Drosha and Dicer with respect to the site of cleavage. Variability at the 5′ and 3′ ends of miRNA and miRNA* may also be due to mismatches in the stem structures of the pri-miRNA and pre-miRNA. The mismatches of the stem strands may lead to a population of different hairpin structures. Variability in the stem structures may also lead to variability in the products of cleavage by Drosha and Dicer.

Nucleic Acids

Nucleic acids are provided herein. The nucleic acids comprise the sequences of SEQ ID NOS: 1-96 or variants thereof. The variant may be a complement of the referenced nucleotide sequence. The variant may also be a nucleotide sequence that is substantially identical to the referenced nucleotide sequence or the complement thereof. The variant may also be a nucleotide sequence which hybridizes under stringent conditions to the referenced nucleotide sequence, complements thereof, or nucleotide sequences substantially identical thereto.

The nucleic acid may have a length of from about 10 to about 250 nucleotides. The nucleic acid may have a length of at least 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 125, 150, 175, 200 or 250 nucleotides. The nucleic acid may be synthesized or expressed in a cell (in vitro or in vivo) using a synthetic gene described herein. The nucleic acid may be synthesized as a single strand molecule and hybridized to a substantially complementary nucleic acid to form a duplex. The nucleic acid may be introduced to a cell, tissue or organ in a single- or double-stranded form or capable of being expressed by a synthetic gene using methods well known to those skilled in the art, including as described in U.S. Pat. No. 6,506,559 which is incorporated by reference.

Nucleic Acid Complexes

The nucleic acid may further comprise one or more of the following: a peptide, a protein, a RNA-DNA hybrid, an antibody, an antibody fragment, a Fab fragment, and an aptamer.

Pri-miRNA

The nucleic acid may comprise a sequence of a pri-miRNA or a variant thereof. The pri-miRNA sequence may comprise from 45-30,000,50-25,000,100-20,000, 1,000-1,500 or 80-100 nucleotides. The sequence of the pri-miRNA may comprise a pre-miRNA, miRNA and miRNA*, as set forth herein, and variants thereof. The sequence of the pri-miRNA may comprise any of the sequences of SEQ ID NOS: 1-96 or variants thereof.

The pri-miRNA may comprise a hairpin structure. The hairpin may comprise a first and a second nucleic acid sequence that are substantially complimentary. The first and second nucleic acid sequence may be from 37-50 nucleotides. The first and second nucleic acid sequence may be separated by a third sequence of from 8-12 nucleotides. The hairpin structure may have a free energy of less than −25 Kcal/mole as calculated by the Vienna algorithm with default parameters, as described in Hofacker et al., Monatshefte f. Chemie 125: 167-188 (1994), the contents of which are incorporated herein by reference. The hairpin may comprise a terminal loop of 4-20, 8-12 or 10 nucleotides. The pri-miRNA may comprise at least 19% adenosine nucleotides, at least 16% cytosine nucleotides, at least 23% thymine nucleotides and at least 19% guanine nucleotides.

Pre-miRNA

The nucleic acid may also comprise a sequence of a pre-miRNA or a variant thereof. The pre-miRNA sequence may comprise from 45-90, 60-80 or 60-70 nucleotides. The sequence of the pre-miRNA may comprise a miRNA and a miRNA* as set forth herein. The sequence of the pre-miRNA may also be that of a pri-miRNA excluding from 0-160 nucleotides from the 5′ and 3′ ends of the pri-miRNA. The sequence of the pre-miRNA may comprise the sequence of SEQ ID NOS: 1-96 or variants thereof.

miRNA

The nucleic acid may also comprise a sequence of a miRNA (including miRNA*) or a variant thereof. The miRNA sequence may comprise from 13-33, 18-24 or 21-23 nucleotides. The miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the miRNA may be the first 13-33 nucleotides of the pre-miRNA. The sequence of the miRNA may also be the last 13-33 nucleotides of the pre-miRNA. The sequence of the miRNA may comprise the sequence of SEQ ID NOS: 1-96 or variants thereof.

Probes

A probe is also provided comprising a nucleic acid described herein. Probes may be used for screening and diagnostic methods, as outlined below. The probe may be attached or immobilized to a solid substrate, such as a biochip.

The probe may have a length of from 8 to 500, 10 to 100 or 20 to 60 nucleotides. The probe may also have a length of at least 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 120, 140, 160, 180, 200, 220, 240, 260, 280 or 300 nucleotides. The probe may further comprise a linker sequence of from 10-60 nucleotides.

Biochip

A biochip is also provided. The biochip may comprise a solid substrate comprising an attached probe or plurality of probes described herein. The probes may be capable of hybridizing to a target sequence under stringent hybridization conditions. The probes may be attached at spatially defined addresses on the substrate. More than one probe per target sequence may be used, with either overlapping probes or probes to different sections of a particular target sequence. The probes may be capable of hybridizing to target sequences associated with a single disorder appreciated by those in the art. The probes may either be synthesized first, with subsequent attachment to the biochip, or may be directly synthesized on the biochip.

The solid substrate may be a material that may be modified to contain discrete individual sites appropriate for the attachment or association of the probes and is amenable to at least one detection method. Representative examples of substrates include glass and modified or functionalized glass, plastics (including acrylics, polystyrene and copolymers of styrene and other materials, polypropylene, polyethylene, polybutylene, polyurethanes, TeflonJ, etc.), polysaccharides, nylon or nitrocellulose, resins, silica or silica-based materials including silicon and modified silicon, carbon, metals, inorganic glasses and plastics. The substrates may allow optical detection without appreciably fluorescing.

The substrate may be planar, although other configurations of substrates may be used as well. For example, probes may be placed on the inside surface of a tube, for flow-through sample analysis to minimize sample volume. Similarly, the substrate may be flexible, such as flexible foam, including closed cell foams made of particular plastics.

The biochip and the probe may be derivatized with chemical functional groups for subsequent attachment of the two. For example, the biochip may be derivatized with a chemical functional group including, but not limited to, amino groups, carboxyl groups, oxo groups or thiol groups. Using these functional groups, the probes may be attached using functional groups on the probes either directly or indirectly using a linker. The probes may be attached to the solid support by either the 5′ terminus, 3′ terminus, or via an internal nucleotide.

The probe may also be attached to the solid support non-covalently. For example, biotinylated oligonucleotides can be made, which may bind to surfaces covalently coated with streptavidin, resulting in attachment. Alternatively, probes may be synthesized on the surface using techniques such as photopolymerization and photolithography.

Diagnostics

As used herein the term “diagnosing” refers to classifying pathology, or a symptom, determining a severity of the pathology (grade or stage), monitoring pathology progression, forecasting an outcome of pathology and/or prospects of recovery.

As used herein the phrase “subject in need thereof” refers to an animal or human subject who is known to have cancer, at risk of having cancer [e.g., a genetically predisposed subject, a subject with medical and/or family history of cancer, a subject who has been exposed to carcinogens, occupational hazard, environmental hazard] and/or a subject who exhibits suspicious clinical signs of cancer [e.g., blood in the stool or melena, unexplained pain, sweating, unexplained fever, unexplained loss of weight up to anorexia, changes in bowel habits (constipation and/or diarrhea), tenesmus (sense of incomplete defecation, for rectal cancer specifically), anemia and/or general weakness]. Additionally or alternatively, the subject in need thereof can be a healthy human subject undergoing a routine well-being check up.

Analyzing presence of malignant or pre-malignant cells can be effected in-vivo or ex-vivo, whereby a biological sample (e.g., biopsy) is retrieved. Such biopsy samples comprise cells and may be an incisional or excisional biopsy. Alternatively the cells may be retrieved from a complete resection.

While employing the present teachings, additional information may be gleaned pertaining to the determination of treatment regimen, treatment course and/or to the measurement of the severity of the disease.

As used herein the phrase “treatment regimen” refers to a treatment plan that specifies the type of treatment, dosage, schedule and/or duration of a treatment provided to a subject in need thereof (e.g., a subject diagnosed with a pathology). The selected treatment regimen can be an aggressive one which is expected to result in the best clinical outcome (e.g., complete cure of the pathology) or a more moderate one which may relieve symptoms of the pathology yet results in incomplete cure of the pathology. It will be appreciated that in certain cases the treatment regimen may be associated with some discomfort to the subject or adverse side effects (e.g., damage to healthy cells or tissue). The type of treatment can include a surgical intervention (e.g., removal of lesion, diseased cells, tissue, or organ), a cell replacement therapy, an administration of a therapeutic drug (e.g., receptor agonists, antagonists, hormones, chemotherapy agents) in a local or a systemic mode, an exposure to radiation therapy using an external source (e.g., external beam) and/or an internal source (e.g., brachytherapy) and/or any combination thereof. The dosage, schedule and duration of treatment can vary, depending on the severity of pathology and the selected type of treatment, and those of skills in the art are capable of adjusting the type of treatment with the dosage, schedule and duration of treatment.

A method of diagnosis is also provided. The method comprises detecting an expression level of a specific cancer-associated nucleic acid in a biological sample. The sample may be derived from a patient. Diagnosis of a specific cancer state in a patient may allow for prognosis and selection of therapeutic strategy. Further, the developmental stage of cells may be classified by determining temporarily expressed specific cancer-associated nucleic acids.

In situ hybridization of labeled probes to tissue arrays may be performed. When comparing the fingerprints between individual samples the skilled artisan can make a diagnosis, a prognosis, or a prediction based on the findings. It is further understood that the nucleic acid sequence which indicate the diagnosis may differ from those which indicate the prognosis and molecular profiling of the condition of the cells may lead to distinctions between responsive or refractory conditions or may be predictive of outcomes.

Kits

A kit is also provided and may comprise a nucleic acid described herein together with any or all of the following: assay reagents, buffers, probes and/or primers, and sterile saline or another pharmaceutically acceptable emulsion and suspension base. In addition, the kits may include instructional materials containing directions (e.g., protocols) for the practice of the methods described herein. The kit may further comprise a software package for data analysis of expression profiles.

For example, the kit may be a kit for the amplification, detection, identification or quantification of a target nucleic acid sequence. The kit may comprise a poly (T) primer, a forward primer, a reverse primer, and a probe.

Any of the compositions described herein may be comprised in a kit. In a non-limiting example, reagents for isolating miRNA, labeling miRNA, and/or evaluating a miRNA population using an array are included in a kit. The kit may further include reagents for creating or synthesizing miRNA probes. The kits will thus comprise, in suitable container means, an enzyme for labeling the miRNA by incorporating labeled nucleotide or unlabeled nucleotides that are subsequently labeled. It may also include one or more buffers, such as reaction buffer, labeling buffer, washing buffer, or a hybridization buffer, compounds for preparing the miRNA probes, components for in situ hybridization and components for isolating miRNA. Other kits of the invention may include components for making a nucleic acid array comprising miRNA, and thus, may include, for example, a solid support.

The following examples are presented in order to more fully illustrate some embodiments of the invention. They should, in no way be construed, however, as limiting the broad scope of the invention.

### EXAMPLES

### Methods

### 1. Tumor Samples

Tumor samples were obtained from several sources. Institutional review approvals were obtained for all samples in accordance with each institute's IRB or IRB-equivalent guidelines. For formalin fixed paraffin-embedded (FFPE) samples, initial diagnosis, histological type, grade and tumor percentages were determined by a pathologist on hematoxilin-eosin (H&E) stained slides, performed on the first and/or last sections of the sample. Samples included primary tumors, metastatic tumors, and two samples of benign prostatic hyperplasia samples (BPH) which showed similar expression profile to prostate tumor samples (not shown). Non-defined samples were not included in this study. Tumor content in 90% of the FFPE samples was above 50%.

### 2. RNA Extraction

For frozen tissue, a sample approximately 0.5 cm³ in dimension was used for RNA extraction. Total RNA was extracted using the miRvana miRNA isolation kit (Ambion) according to the manufacturer's instructions. Briefly, the sample is homogenized in a denaturing lysis solution followed by an acid-phenol:chloroform extraction. Finally, the sample is purified on a glass-fiber filter.

For FFPE samples, total RNA was isolated from seven to ten 10-μm-thick tissue sections using the miRdictor™ extraction protocol developed at Rosetta Genomics. Briefly, the sample is incubated few times in Xylene at 57° C. to remove paraffin excess, followed by Ethanol washes. Proteins are degraded by proteinase K solution at 45° C. for a few hours. The RNA is extracted with acid phenol:chloroform followed by ethanol precipitation and DNAse digestion. Total RNA quantity and quality is checked by spectrophotometer (Nanodrop ND-1000).

3. miRdicator™ Array Platform

Custom microarrays were produced by printing DNA oligonucleotide probes to 688 human microRNAs. Each probe, printed in triplicate, carries up to 22-nucleotide (nt) linker at the 3′ end of the microRNA's complement sequence in addition to an amine group used to couple the probes to coated glass slides. 20 μM of each probe were dissolved in 2×SSC+0.0035% SDS and spotted in triplicate on Schott Nexterion® Slide E coated microarray slides using a Genomic Solutions® BioRobotics MicroGrid II according the MicroGrid manufacturer's directions. 54 negative control probes were designed using the sense sequences of different microRNAs. Two groups of positive control probes were designed to hybridize to miRdicator™ array (i) synthetic small RNA were spiked to the RNA before labeling to verify the labeling efficiency and (ii) probes for abundant small RNA (e.g. small nuclear RNAs (U43, U49, U24, Z30, U6, U48, U44), 5.8 s and 5 s ribosomal RNA) are spotted on the array to verify RNA quality. The slides were blocked in a solution containing 50 mM ethanolamine, 1M Tris (pH9.0) and 0.1% SDS for 20 min at 50° C., then thoroughly rinsed with water and spun dry.

4. Cy-Dye Labeling of miRNA for miRdicator™ Array

Five μg of total RNA were labeled by ligation (Thomson et al., Nature Methods 2004, 1:47-53) of an RNA-linker, p-rCrU-Cy/dye (Dharmacon), to the 3′-end with Cy3 or Cy5. The labeling reaction contained total RNA, spikes (0.1-20 fmoles), 300 ng RNA-linker-dye, 15% DMSO, 1× ligase buffer and 20 units of T4 RNA ligase (NEB) and proceeded at 4° C. for 1 hr followed by 1 hr at 37° C. The labeled RNA was mixed with 3× hybridization buffer (Ambion), heated to 95° C. for 3 min and than added on top of the miRdicator™ array. Slides were hybridized 12-16 hr in 42° C., followed by two washes in room temperature with 1×SSC and 0.2% SDS and a final wash with 0.1×SSC.

Arrays were scanned using an Agilent Microarray Scanner Bundle G2565BA (resolution of 10 μm at 100% power). Array images were analyzed using SpotReader software (Niles Scientific).

### 5. Array Signal Calculation and Normalization

Triplicate spots were combined to produce one signal for each probe by taking the logarithmic mean of reliable spots. All data was log-transformed (natural base) and the analysis was performed in log-space. A reference data vector for normalization R was calculated by taking the median expression level for each probe across all samples. For each sample data vector S, a 2nd degree polynomial F was found so as to provide the best fit between the sample data and the reference data, such that R≈F(S). Remote data points (“outliers”) were not used for fitting the polynomial F. For each probe in the sample (element Si in the vector S), the normalized value (in log-space) Mi is calculated from the initial value Si by transforming it with the polynomial function F, so that Mi=F(Si). Data in FIGS. 3A, B was translated back to linear-space (by taking the exponent). Using only the training set samples to generate the reference data vector did not affect the results.

### 6. Logistic Regression

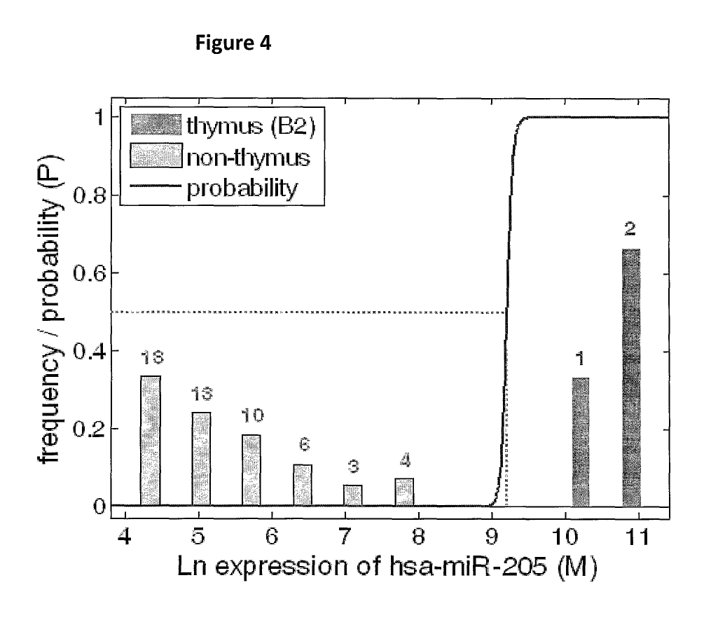

The aim of a logistic regression model is to use several features, such as expression levels of several microRNAs, to assign a probability of belonging to one of two possible groups, such as two branches of a node in a binary decision-tree. Logistic regression models the natural log of the odds ratio, i.e. the ratio of the probability of belonging to the first group, for example the left branch in a node of a binary decision-tree (P) over the probability of belonging to the second group, for example the right branch in such a node (1-P), as a linear combination of the different expression levels (in log-space). The logistic regression assumes that:

$$ln ( \frac{P}{1 - P} ) = {β}_{0} + {\sum }_{i = 1}^{N} {β}_{i} \cdot {M}_{i} = {β}_{0} + {β}_{1} \cdot {M}_{1} + {β}_{2} \cdot {M}_{2} + … ,$$

where β₀ is the bias, Mᵢ is the expression level (normalized, in log-space) of the i-th microRNA used in the decision node, and βᵢ is its corresponding coefficient. βi>0 indicates that the probability to take the left branch (P) increases when the expression level of this microRNA (Mi) increases, and the opposite for βi<0. If a node uses only a single microRNA (M), then solving for P results in (FIG. 4):

$$P = \frac{{e}^{{β}_{0} + {β}_{1} \cdot M}}{1 + {e}^{{β}_{0} + {β}_{1} \cdot M}} .$$

The regression error on each sample is the difference between the assigned probability P and the true “probability” of this sample, i.e. 1 if this sample is in the left branch group and 0 otherwise. The training and optimization of the logistic regression model calculates the parameters β and the p-values (for each microRNA by the Wald statistic and for the overall model by the χ2 (chi-square) difference), maximizing the likelihood of the data given the model and minimizing the total regression error

$${\sum }_{{Samples}_{{i n}_{{first}_{group}}}} ( 1 - {P}_{j} ) + {\sum }_{{Samples}_{{i n}_{{second}_{group}}}} {P}_{j} .$$

The probability output of the logistic model is here converted to a binary decision by comparing P to a threshold, denoted by P_{TH}, i.e. if P>P_{TH} then the sample belongs to the left branch (“first group”) and vice versa. Choosing at each node the branch which has a probability>0.5, i.e. using a probability threshold of 0.5, leads to a minimization of the sum of the regression errors. However, as the goal was the minimization of the overall number of misclassifications (and not of their probability), a modification which adjusts the probability threshold (P_{TH}) was used in order to minimize the overall number of mistakes at each node (Table 2). For each node the threshold to a new probability threshold P_{TH} was optimized such that the number of classification errors is minimized. This change of probability threshold is equivalent (in terms of classifications) to a modification of the bias β₀, which may reflect a change in the prior frequencies of the classes.

### 7. Stepwise Logistic Regression and Feature Selection

The original data contains the expression levels of hundreds of microRNAs for each sample, i.e. hundreds of data features. In training the classifier for each node, only a small subset of these features was selected and used for optimizing a logistic regression model. In the initial training this was done using a forward stepwise scheme. The features were sorted in order of decreasing log-likelihoods, and the logistic model was started off and optimized with the first feature. The second feature was then added, and the model re-optimized. The regression error of the two models was compared: if the addition of the feature did not provide a significant advantage (a χ2 difference less than 7.88, p-value of 0.005), the new feature was discarded. Otherwise, the added feature was kept. Adding a new feature may make a previous feature redundant (e.g. if they are very highly correlated). To check for this, the process iteratively checks if the feature with lowest likelihood can be discarded (without losing χ2 difference as above). After ensuring that the current set of features is compact in this sense, the process continues to test the next feature in the sorted list, until features are exhausted. No limitation on the number of feature was inserted into the algorithm but in most cases 2-3 features were selected.

The stepwise logistic regression method was used on subsets of the training set samples by re-sampling the training set with repetition (“bootstrap”) so that each of the 23 runs contained about two-thirds of the samples at least once, and any one sample had >99% chance of being left out at least once. This resulted in an average of 2˜3 features per node (4˜8 in more difficult nodes). We selected a robust set of 2˜3 features per each node (Table 2) by comparing features that were repeatedly chosen in the bootstrap sets to previous evidence, and considering their signal strengths and reliability. When using these selected features to construct the classifier, the stepwise process was not used and the training optimized the logistic regression model parameters only.

### 8. Restriction of Classes by Gender and Liver Metastases

The decision-tree framework allows easy implementation of available clinical information into the classification. Two such data are used: gender and liver metastases. Samples from female patients were not allowed to be classified as originating from testis or prostate; thus, samples of female patients that reached node #2 were automatically classified to the right branch, and likewise the left branch (=breast) at node #17. Samples from male patients were not allowed to be classified as originating from endometrium or ovary, and were automatically classified to the left branch at node 20. Samples that were indicated as liver metastases were not allowed to be classified as originating from liver tissue and were classified to the right branch in node #1. Thus, additional information is easily utilized without loss of generality or need to retrain the classifier.

### 9. K-Nearest-Neighbors (KNN) Classification Algorithm

The KNN algorithm (see e.g. Ma et al., Arch Pathol Lab Med 2006, 130:465-73) calculated the distance (Pearson correlation) of any sample to all samples in the training set, and classifies the sample by the majority vote of the k samples which are most similar (k being a parameter of the classifier). The correlation is calculated on a pre-defined set of microRNAs (data features), selected by going over all pairs of tissue types (classes) and collecting microRNAs that were significantly differentially expressed between any two classes. Using only the intersection of this list with the 48 microRNAs that were used by the decision-tree did not reduce the performance, highlighting the information content of these microRNAs. KNN algorithms with k=1, 3, 5 were compared, and the optimal performer was selected, using k=3 and the smaller set of microRNAs.

10. qRT-PCR

1 μg of total RNA is subjected to polyadenylation reaction as described before (Shi and Chiang, BioTechniques 2005, 39:519-525). Briefly, RNA is incubated in the presence of poly (A) polymerase (PAP) (Takara-2180A), MnCl2, and ATP for 1 h at 37° C. Reverse transcription is performed on the total RNA. An oligodT primer harboring a consensus sequence (complementary to the reverse primer, oligodT starch, an N nucleotide (a mixture of all A, C, and G) and V nucleotide (mixture of 4 nucleotides) is used for reverse transcription reaction. The primer is first annealed to the polyA-RNA and than subjected to a reverse transcription reaction of SuperScript II RT (Invitrogen). The cDNA is than amplified by real time PCR reaction, using a microRNA specific forward primer, TaqMan probe and universal reverse primer that is complementary to the 3′ sequence of the oligo dT tail. The reactions are incubated for 10 min. at 95° C. followed by 42 cycles of 95° C. for 15 sec and 60° C. for 1 min.

FIG. 3C shows data normalized to U6 snRNA (see e.g. Thompson et al., Genes & Development 2006, 20:2202-2207). Data in FIG. 3D was normalized by U6, transformed to linear space (by the exponent base 2), and multiplied by a constant (59,000) to shift numeric values to have the same median value as the array signals. Comparing the distributions of the three microRNAs in the two separate sample subsets (six groups in all) between the microarray and the qRT-PCR data, we obtained a mean Kolmogorov-Smirnov statistic of 0.32. Only two (of the six) groups had significantly different distributions (KS-statistic<0.05), most groups were not significantly different by the Kolmogorov-Smirnov test.

### Example 1

### Samples and Profiling

Since formalin-fixed paraffin-embedded (FFPE) archival samples are an important source for tumor material, we developed a method for extracting RNA from FFPE blocks which preserves the microRNA fraction. We compared RNA extracted from fresh-frozen, formalin-fixed, or FFPE samples, and demonstrated that the RNA quantity and quality was similar for all preservation methods. Furthermore, the microRNA profile was stable in FFPE samples for as long as 11 years of storage.

MicroRNA profiling was performed on Rosetta Genomics' miRdicator™ microarrays¹⁹, containing probes for all microRNA in miRBase (version 9)³.

333 FFPE samples and 3 fresh-frozen samples were collected and profiled, including 205 primary tumors and 131 metastatic tumors, representing 22 different tumor origins or “classes” (see Table 1 for a summary of samples). Tumor percentage was at least 50% for more than 90% of the samples. 83 of the samples (approximately 25% of each class) were randomly selected as a blinded test set. 65 additional primary tumor samples (53 FFPE and 12 fresh-frozen samples) were profiled only on qRT-PCR as a validation for selected microRNAs. Overall, 401 samples were included in this study.

### Example 2

### Comparison of Primary and Metastatic Tumors

Due to the difficulty of obtaining sufficient numbers of metastatic samples, this study has relied on primary tumors to augment the sample set. Differences in expression profiles between primary and metastatic samples can be expected because of underlying biological differences in the tumors, or because of contamination from neighboring tissues. Such effects can hinder the performance of tumor classifiers on metastatic samples.

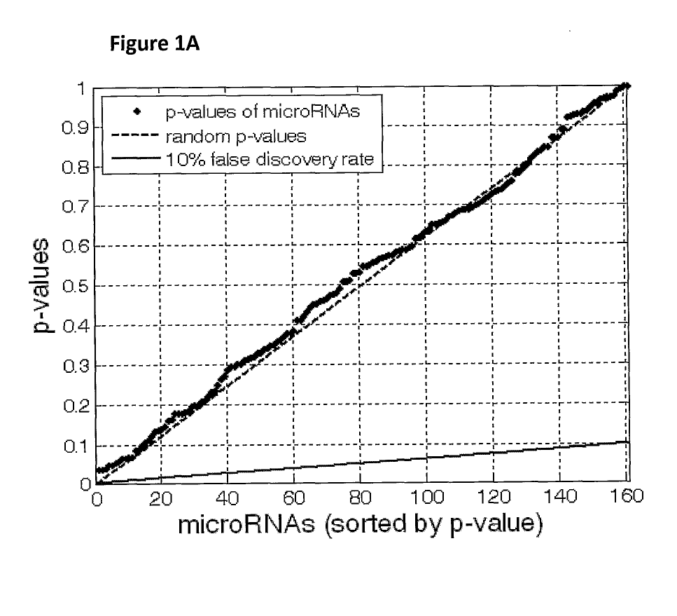

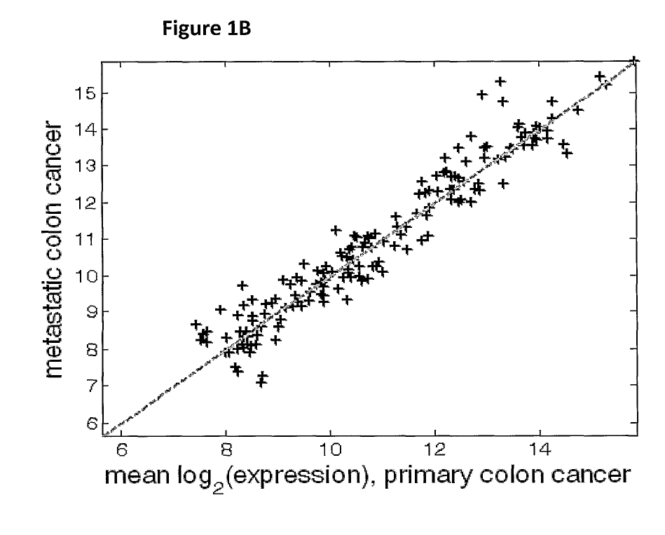

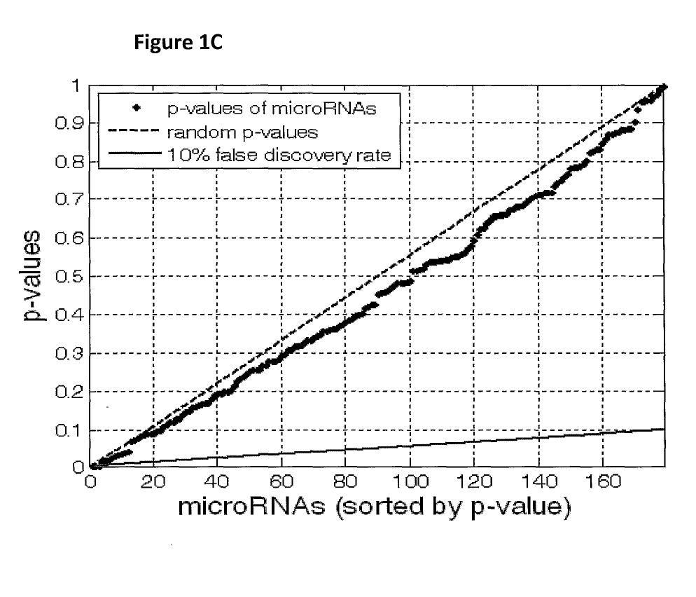

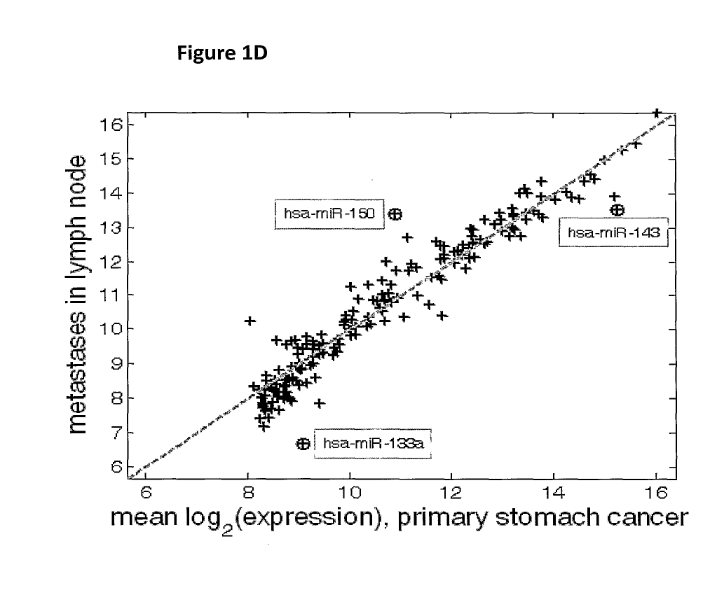

For most tissue origins, such as breast cancer or colon cancer (FIGS. 1A, B), no significant differences between primary and metastatic tumors were found. In other cases, a small set of microRNAs were differentially expressed. For example, in comparing stomach primary tumor samples to samples of stomach metastases to the lymph node, 3 microRNAs were significantly differentially expressed (FIGS. 1C, D). Hsa-miR-143 (SEQ ID NO: 99), characteristic of epithelial layers⁵, and hsa-miR-133a (SEQ ID NO: 97), which is characteristic of muscle tissue², were over-expressed in the primary tumors taken from the stomach; in contrast, hsa-miR-150 (SEQ ID NO: 101), which was previously identified as highly expressed in lymphocytes²⁰, was present at higher levels in the metastatic samples taken from the lymph-node. In addition, samples from primary tumors such as prostate or head and neck, which often contain surrounding muscle tissue, showed significant expression levels of miR-1, miR-206, and miR-133a, microRNAs that are specific to skeletal muscle². We concluded that primary tumors can be used in training a classifier for metastases, but must be used with care and with attention to specific markers and to context. To reduce potential biases from these effects, we minimized the use of microRNAs in nodes where cross-contamination may have confounding effects—e.g., muscle-related microRNAs (miR-11133/206) and hsa-miR-150 were not used.

### Example 3

### Decision-Tree Classification Algorithm

A tumor classifier was built using the microRNA expression levels by applying a binary tree classification scheme (FIG. 2). This framework is set up to utilize the specificity of microRNAs in tissue differentiation and embryogenesis: different microRNAs are involved in various stages of tissue specification, and are used by the algorithm at different decision points or “nodes”. The tree breaks up the complex multi-tissue classification problem into a set of simpler binary decisions. At each node, classes which branch out earlier in the tree are not considered, reducing interference from irrelevant samples and further simplifying the decision (FIG. 3A). The decision at each node can then be accomplished using only a small number of microRNA biomarkers, which have well-defined roles in the classification (Table 2). The structure of the binary tree was based on a hierarchy of tissue development and morphological similarity¹⁸, which was modified by prominent features of the microRNA expression patterns (FIG. 2). For example, the expression patterns of microRNAs indicated a significant difference between lung carcinoid and other lung cancer types, and these are therefore separated at node #12 (FIGS. 3A, B) into separate branches (FIG. 2). Interestingly, an automated algorithm for dividing the data into a binary classification tree generated trees with a similar structure, yet lacked flexibility in structure and in individual node classifiers and resulted in significantly poorer performance.

For each of the individual nodes logistic regression models were used, a robust family of classifiers which are frequently used in epidemiological and clinical studies to combine continuous data features into a binary decision (FIG. 3A, FIG. 4 and Methods). Since gene expression classifiers have an inherent redundancy in selecting the gene features, we used bootstrapping on the training sample set as a method to select a stable microRNA set for each node (Methods). This resulted in a small number (usually 2-3) of microRNA features per node, totaling 48 microRNAs for the full classifier (Table 2). Our approach provides a systematic process for identifying new biomarkers for differential expression.

### Example 4

### Classifier Performance

### Cross Validation and High-Confidence Classifications

As a first step, the performance of the classifier was tested using leave-one-out cross validation (LOOCV) within the training set. LOOCV simulates the performance of a classification algorithm on unseen samples. In LOOCV, the algorithm is repeatedly re-trained, leaving out one sample in each round, and testing each sample on a classifier that was trained without this sample. The decision-tree algorithm reached an average sensitivity, or accuracy, of 78% and specificity of 99%, with significant variation between different classes. The performance was compared to that of the commonly-used K-nearest-neighbors (KNN) classification algorithm^{8,15,18}. The KNN algorithm (at the optimal k=3) showed poorer performance than the tree (71% average sensitivity with equal specificity), with different classes having significant differences in sensitivity between the algorithms.

In clinical practice it is often useful to assess information of different degrees of confidence^{17,18}. In the diagnosis of CUP in particular, a short list of highly probable possibilities is a practical option when no definite diagnosis can be made. Since the decision-tree and the KNN algorithms are designed differently and trained independently, improved accuracy and greater confidence can be obtained by combining and comparing their classifications. The union of the predictions made by the two algorithms included the correct class in 85% of the cases. In 69% of the cases the two algorithms agreed, generating a single, high-confidence prediction. Satisfyingly, 93% of these high-confidence predictions accurately identified the correct class of the sample, with more than half of the 22 tumor classes reaching 100% sensitivity.

### Example 5

### Classifier Performance

### Independent Blinded Test Set

The most important test of a classification algorithm is on a blinded test set. We set aside approximately one quarter of the samples, randomly selected to represent the different classes, as an independent test set, and tested the performance of the classifiers (Table 3). The performance on the test set did not decrease compared to the performance of LOOCV in the training set, a highly desirable feature of a classifier, indicating that the classifier is robust and not over-fit. 86% of the cases were accurately predicted by the union of the two predictors (most classes had 100% sensitivity). Among high confidence predictions, which were two thirds of the cases, 89% were accurately classified. Even in the blinded test set, an overwhelming 16 of the 22 classes had 100% accuracy in the high-confidence prediction. Finally, we checked the performance of the classification on the metastatic samples of the blinded test set. Here, too, the classifier reached 85% sensitivity for high-confidence classifications. The fact that the performance on the blinded metastatic samples was that high supports the approach of augmenting the training set with primary tumors, concomitantly with avoiding potentially confounding markers.

### Example 6

### Validation by an Independent Platform

### qRT-PCR

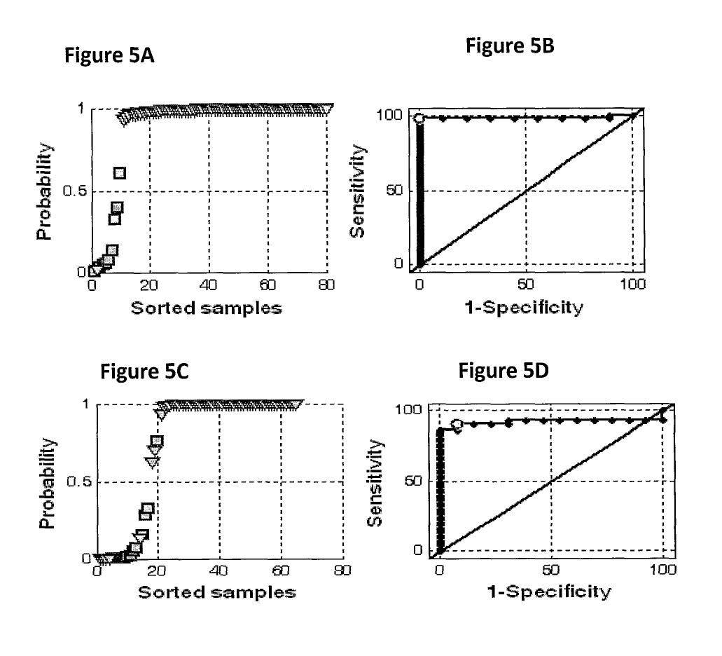

The above decision-tree algorithm which was developed based on an array platform, assigns specific roles to microRNAs in binary decisions between groups of tissues. In order to rule out effects of a specific platform, we validated the significance of a subset of these microRNAs on Rosetta Genomics' miRdicator™ high sensitivity qRT-PCR platform (Methods), using 15 of the original samples plus 65 independent samples. Although the measured signal values differ across platforms, the microRNAs maintain their diagnostic roles (FIGS. 3C, D) and can be used for accurate classification (FIG. 5).

TABLE 1
Cancer types, classes and histology

| Class | Cancer types and histological classifications |
|---|---|
| bladder | Transitional cell carcinoma; Metastasizes (Mets.) to Brain; Mets. to Lung |
| brain | Anaplastic astrocytoma; Low grade astrocytoma; anaplastic |
| | oligodendroglioma; Glioblastoma multiforme; Oligodendroglioma |
| breast | Infiltrating ductal carcinoma; Infiltrating lobular carcinoma; Mucin |
| | producing; Papillary; Mets. to Brain; Mets. to Liver; Mets. to Lung; Mets. |
| | to Lymph Node |
| colon | Adenocarcinoma; Mets. to Brain; Mets. to Liver; Mets. to Lung |
| endometrium | Endometrioid adenocarcinoma; Serous; Mets. to Brain; Mets. to Lymph |
| | Node |
| head & neck* | Squamous cell carcinoma; Mets. to Lung-Pleura; Mets. to Lymph Node |
| kidney | Clear cell carcinoma; Renal cell carcinoma; Mets. to Brain; Mets. to Liver; |
| | Mets. to Lung; Mets. to Lung-Pleura |
| liver | Hepatocellular carcinoma |
| lung | Non-small cell carcinoma; Adenocarcinoma; Squamous cell carcinoma; |
| | Large cell; Neuroendocrine; Small cell; Carcinoid |
| lung pleura | Mesothelioma - epithelioid type; Mesothelioma - sarcomatoid type |
| lymph node | Hodgkin's Lymphoma - classic; Hodgkin's Lymphoma - Nodular |
| | sclerosis; Non-Hodgkin's lymphoma; Diffused large B cell; |
| melanocytes | Malignant melanoma; Mets. to Brain; Mets. to Lung; Mets. to Lymph |
| | Node |
| meninges | Meningioma; Atypical meningioma; |
| ovary | Serous cystadenocarcinoma; Adenocarcinoma; Mets. to Liver; Mets. to |
| | Lung-Pleura; Mets. to Lymph Node |
| pancreas | Exocrine adenocarcinoma; Adenocarcinoma - Mucin producing; |
| | Adenocarcinoma - intraductal; Mets. to Lung |
| prostate | BPH; Adenocarcinoma; Mets. to Lung |
| sarcoma | Ewing sarcoma; Fibrosarcoma; Leiomyosarcoma; Liposarcoma; Malignant |
| | phyllodes tumor; Mixed mullerian tumor; Osteosarcoma; Synovial |
| | sarcoma; Mets. to Brain; Mets. to Lung |
| stomach* | Adenocarcinoma; Mucin producing; Gastroesophageal junction |
| | adenocarcinoma; Mets. to Liver; Mets. to Lymph Node |
| GIST | Gastrointestinal stromal tumor of the small intestine |
| testis | Seminoma |
| thymus | Thymoma - type B2; Thymoma - type B3 |
| thyroid | Papillary carcinoma; Tall cell; Mets. to Lung; Mets. to Lymph Node |

*The “head and neck” class includes cancers of head and neck and squamous carcinoma of esophagus (see FIG. 2).
*The “stomach” class includes both stomach cancers and gastroesophageal junction adenocarcinomas;
“GIST” indicates gastrointestinal stromal tumors.

TABLE 2
Nodes of the decision-tree and microRNAs used in each node

| | | | microRNAs | miR | Hairpin |
|---|---|---|---|---|---|
| | left | right | used at | SEQ ID | SEQ ID |
| node # | branch | branch | the node | NO: | NO: |
| 1^{a} | liver | node #2 | hsa-miR-122a | 1 | 2 |
| | | | hsa-miR-200c† | 3 | 4 |
| 2¹ | testis | node #3 | hsa-miR-372 | 5 | 6 |
| 3 | node #12 | node #4 | hsa-miR-200c | 3 | 4 |
| | | | hsa-miR-181a | 95 | 96 |
| | | | hsa-miR-205 | 7 | 8 |
| 4 | node #5 | node #6 | hsa-miR-146^{a} | 9 | 10 |
| | | | hsa-miR-200a | 11 | 12 |
| | | | hsa-miR-92a | 13 | 14 |
| 5 | lymph | melano- | hsa-miR-142-3p | 15 | 16 |
| | node | cytes | hsa-miR-509 | 17 | 18 |
| 6 | brain | node #7 | hsa-miR-92b | 19 | 20 |
| | | | hsa-miR-9* | 21 | 22 |
| | | | hsa-miR-124a | 23 | 24 |
| 7 | meninges | node #8 | hsa-miR-152 | 25 | 26 |
| | | | hsa-miR-130a | 27 | 28 |
| 8 | thymus (B2) | node #9 | hsa-miR-205 | 7 | 8 |
| 9 | node #11 | node #10 | hsa-miR-192 | 29 | 30 |
| | | | hsa-miR-21 | 31 | 32 |
| | | | hsa-miR-210 | 33 | 34 |
| | | | hsa-miR-34b | 35 | 36 |
| 10 | lung- | kidney | hsa-miR-194 | 37 | 38 |
| | pleura | | hsa-miR-382 | 39 | 40 |
| | | | hsa-miR-210 | 33 | 34 |
| 11 | sarcoma | GIST | hsa-miR-187 | 41 | 42 |
| | | | hsa-miR-29b | 43 | 44 |
| 12 | node #13 | node #16 | hsa-miR-145 | 45 | 46 |
| | | | hsa-miR-194 | 37 | 38 |
| | | | hsa-miR-205 | 7 | 8 |
| 13 | node #14 | lung | hsa-miR-21 | 31 | 32 |
| | | (carcinoid) | hsa-let-7e | 47 | 48 |
| 14 | colon | node #15 | hsa-let-7i | 49 | 50 |
| | | | hsa-miR-29a | 51 | 52 |
| 15 | stomach* | pancreas | hsa-miR-214 | 53 | 54 |
| | | | hsa-miR-19b | 55 | 56 |
| | | | hsa-let-7i | 49 | 50 |
| 16 | node #17 | node #18 | hsa-miR-196a | 57 | 58 |
| | | | hsa-miR-363 | 59 | 60 |
| | | | hsa-miR-31 | 61 | 62 |
| | | | hsa-miR-193a | 63 | 64 |
| | | | hsa-miR-210 | 33 | 34 |
| 17² | breast | prostate | hsa-miR-27b | 65 | 66 |
| | | | hsa-let-7i | 49 | 50 |
| | | | hsa-miR-181b | 67 | 68 |
| 18 | node #19 | node #23 | hsa-miR-205 | 7 | 8 |
| | | | hsa-miR-141 | 69 | 70 |
| | | | hsa-miR-193b | 71 | 72 |
| | | | hsa-miR-373 | 73 | 74 |
| 19 | thyroid | node #20 | hsa-miR-106b | 75 | 76 |
| | | | hsa-let-7i | 49 | 50 |
| | | | hsa-miR-138 | 77 | 78 |
| 20³ | node #21 | node #22 | hsa-miR-10b | 79 | 80 |
| | | | hsa-miR-375 | 81 | 82 |
| | | | hsa-miR-99a | 83 | 84 |
| 21 | lung | bladder | hsa-miR-205 | 7 | 8 |
| | | | hsa-miR-152 | 25 | 26 |
| 22 | endo- | ovary | hsa-miR-345 | 85 | 86 |
| | metrium | | hsa-miR-29c | 87 | 88 |
| | | | hsa-miR-182 | 89 | 90 |
| 23 | thymus (B3) | node #24 | hsa-miR-192 | 29 | 30 |
| | | | hsa-miR-345 | 85 | 86 |
| 24 | lung | head & | hsa-miR-182 | 89 | 90 |
| | (squamous) | neck* | hsa-miR-34a | 91 | 92 |
| | | | hsa-miR-148b | 93 | 94 |

†Hsa-miR-200c and hsa-miR-141 are part of one predicted polycistronic pri-miR⁶ and are very similarly expressed. These two microRNAs can be used interchangeably in the tree with very slight effect on the results. Hsa-miR-200c had slightly better performance (in the training set) in node #1.
^{a}For samples indicated as metastasis to the liver, classification proceeds to the right branch at this node and continues to node #3.
¹For samples indicated as originating from a female patient, classification proceeds to the right branch at this node and continues to node #3.
²For samples indicated as originating from a female patient, classification proceeds to the left branch at this node and is classified as breast.
³For samples is indicated as originating from a male patient, classification proceeds to the left branch at this node and continues to node #21.

The “stomach*” class includes both stomach cancers and gastroesophageal junction adenocarcinomas; the “head and neck*” class includes cancers of head and neck and squamous carcinoma of esophagus (see FIG. 2). “GIST” indicates gastrointestinal stromal tumors.

In the decision-tree scheme, some microRNAs separate large sections of the tree and decide between two branches that lead to further nodes; and other nodes separate at terminal nodes where at least one of the two branches leads to a specific tissue type. An implication of the tree design is that microRNAs that separate between two branches can also be used to separate between any two single tissue types that are “leaves” of the two alternative branches of this node. For example, at node #12, hsa-miR-194 separates between the branch leading to node #13 and the branch leading to node #16. Since “colon” is an indirect leaf of node #13 (through node #14), and “breast” is an indirect leaf of node #16 (through node #17), this implies that hsa-miR-194 can also be used to separate between “colon” and “breast” in the absence of other tissue types.

Table 3 shows the number of samples in the training and test sets and the performance of classification on the blinded test set, for each class separately and overall averaged over all samples. “Sens” indicates sensitivity, “Spec” indicates specificity. “Tree” refers to the decision-tree algorithm; “Union” is the one/two answers that are obtained by collecting the predictions of both the decision-tree and KNN algorithms. “High conf. Frac” is the fraction of the samples with high confidence predictions, for which both the decision-tree and KNN algorithms agree on the classification. “High conf. Sens” is the sensitivity among the high confidence predictions. The last columns show performance on the subset of the test set which are metastatic cancer samples. The “stomach*” class includes both stomach cancers and gastroesophageal junction adenocarcinomas; the “head and neck*” class includes cancers of head and neck and squamous carcinoma of esophagus (see FIG. 2). “GIST” indicates gastrointestinal stromal tumors.

TABLE 3
Performance of classification on blinded test set

| | Samples | | Results on blinded test set (%) | | | | | | Metastases in test set | | | |
|---|---|---|---|---|---|---|---|---|---|---|---|---|
| | N | N | Tree | Tree | KNN | Union | High | conf. | | Union | High | conf. |
| | Train | Test | Sens | Spec | Sens | Sens | Frac | Sens | N | Sens | Frac | Sens |
| bladder | 4 | 2 | 0 | 100 | 0 | 0 | 100 | 0 | 1 | 0 | 100 | 0 |
| brain | 10 | 5 | 100 | 100 | 100 | 100 | 100 | 100 | 0 | | | |
| breast | 19 | 5 | 60 | 97 | 60 | 60 | 80 | 75 | 4 | 50 | 75 | 67 |
| colon | 15 | 5 | 40 | 99 | 40 | 60 | 60 | 33 | 3 | 100 | 33 | 100 |
| endometrium | 7 | 3 | 0 | 99 | 67 | 67 | 0 | | 1 | 100 | 0 | |
| head & neck* | 23 | 8 | 100 | 99 | 88 | 100 | 88 | 100 | 0 | | | |
| kidney | 15 | 5 | 100 | 99 | 80 | 100 | 80 | 100 | 2 | 100 | 50 | 100 |
| liver | 4 | 2 | 100 | 99 | 50 | 100 | 50 | 100 | 0 | | | |
| lung | 44 | 5 | 80 | 95 | 100 | 100 | 80 | 100 | 1 | 100 | 100 | 100 |
| lung-pleura | 5 | 2 | 50 | 99 | 50 | 50 | 50 | 100 | 0 | | | |
| lymph-node | 10 | 5 | 60 | 100 | 40 | 80 | 40 | 50 | 0 | | | |
| melanocytes | 21 | 5 | 60 | 97 | 80 | 80 | 60 | 100 | 4 | 75 | 50 | 100 |
| meninges | 6 | 3 | 100 | 99 | 100 | 100 | 100 | 100 | 0 | | | |
| ovary | 10 | 4 | 75 | 97 | 75 | 100 | 50 | 100 | 1 | 100 | 100 | 100 |
| pancreas | 6 | 2 | 50 | 100 | 50 | 100 | 0 | | 0 | | | |
| prostate | 6 | 2 | 100 | 100 | 100 | 100 | 100 | 100 | 0 | | | |
| sarcoma | 15 | 5 | 40 | 99 | 80 | 80 | 40 | 100 | 4 | 75 | 50 | 100 |
| stomach* | 13 | 7 | 71 | 96 | 57 | 86 | 43 | 100 | 1 | 100 | 100 | 100 |
| stromal | 5 | 2 | 100 | 100 | 100 | 100 | 100 | 100 | 0 | | | |
| testis | 2 | 1 | 100 | 100 | 100 | 100 | 100 | 100 | 0 | | | |
| thymus | 5 | 2 | 100 | 98 | 50 | 100 | 50 | 100 | 0 | | | |
| thyroid | 8 | 3 | 100 | 100 | 100 | 100 | 100 | 100 | 0 | | | |
| Overall | 253 | 83 | 72 | 99 | 72 | 86 | 66 | 89 | 22 | 77 | 59 | 85 |

For some of the microRNAs in Table 2, other variant microRNAs are known in the human genome that have similar seed sequence (identical nucleotides 2-8) (see Table 4), and therefore are considered to target very similar set of (mRNA-coding) genes (via the RISC machinery). These microRNAs with identical seed sequence may be substituted for the indicated miRs.

TABLE 4
microRNAs with identical seed sequence

| | | | | |
|---|---|---|---|---|
| Indicated | | miRs with | | SEQ |
| miRs | Seed | same seed | miR sequence | ID# |

| | | | | |
|---|---|---|---|---|
| hsa-let-7e | GAGGTAG | hsa-let-7a | TGAGGTAGTAGGTTGTATAGTT | 103 |
| | GAGGTAG | hsa-let-7b | TGAGGTAGTAGGTTGTGTGGTT | 104 |
| | GAGGTAG | hsa-let-7c | TGAGGTAGTAGGTTGTATGGTT | 105 |
| | GAGGTAG | hsa-let-7d | AGAGGTAGTAGGTTGCATAGTT | 106 |
| | GAGGTAG | hsa-let-7f | TGAGGTAGTAGATTGTATAGTT | 107 |
| | GAGGTAG | hsa-let-7g | TGAGGTAGTAGTTTGTACAGTT | 108 |
| | GAGGTAG | hsa-let-7i | TGAGGTAGTAGTTTGTGCTGTT | 49 |
| | GAGGTAG | hsa-miR-98 | TGAGGTAGTAAGTTGTATTGTT | 109 |
| hsa-let-7i | GAGGTAG | hsa-let-7a | TGAGGTAGTAGGTTGTATAGTT | 103 |
| | GAGGTAG | hsa-let-7b | TGAGGTAGTAGGTTGTGTGGTT | 104 |
| | GAGGTAG | hsa-let-7c | TGAGGTAGTAGGTTGTATGGTT | 105 |
| | GAGGTAG | hsa-let-7d | AGAGGTAGTAGGTTGCATAGTT | 106 |
| | GAGGTAG | hsa-let-7e | TGAGGTAGGAGGTTGTATAGTT | 47 |
| | GAGGTAG | hsa-let-7f | TGAGGTAGTAGATTGTATAGTT | 107 |
| | GAGGTAG | hsa-let-7g | TGAGGTAGTAGTTTGTACAGTT | 108 |
| | GAGGTAG | hsa-miR-98 | TGAGGTAGTAAGTTGTATTGTT | 109 |
| hsa-miR-106b | AAAGTGC | hsa-miR-106a | AAAAGTGCTTACAGTGCAGGTAG | 165 |
| | AAAGTGC | hsa-miR-17 | CAAAGTGCTTACAGTGCAGGTAG | 110 |
| | AAAGTGC | hsa-miR-20a | TAAAGTGCTTATAGTGCAGGTAG | 111 |
| | AAAGTGC | hsa-miR-20b | CAAAGTGCTCATAGTGCAGGTAG | 112 |
| | AAAGTGC | hsa-miR-519d | CAAAGTGCCTCCCTTTAGAGTG | 113 |
| | AAAGTGC | hsa-miR-526b* | GAAAGTGCTTCCTTTTAGAGGC | 114 |
| | AAAGTGC | hsa-miR-93 | CAAAGTGCTGTTCGTGCAGGTAG | 115 |
| hsa-miR-10b | ACCCTGT | hsa-miR-10a | TACCCTGTAGATCCGAATTTGTG | 116 |
| hsa-miR-124 | AAGGCAC | hsa-miR-506 | TAAGGCACCCTTCTGAGTAGA | 117 |
| hsa-miR-130a | AGTGCAA | hsa-miR-130b | CAGTGCAATGATGAAAGGGCAT | 118 |
| | AGTGCAA | hsa-miR-301a | CAGTGCAATAGTATTGTCAAAGC | 119 |
| | AGTGCAA | hsa-miR-301b | CAGTGCAATGATATTGTCAAAGC | 120 |
| | AGTGCAA | hsa-miR-454 | TAGTGCAATATTGCTTATAGGGT | 121 |
| hsa-miR-141 | AACACTG | hsa-miR-200a | TAACACTGTCTGGTAACGATGT | 11 |
| hsa-miR-146a | GAGAACT | hsa-miR-146b-5p | TGAGAACTGAATTCCATAGGCT | 122 |
| hsa-miR-148b | CAGTGCA | hsa-miR-148a | TCAGTGCACTACAGAACTTTGT | 123 |
| | CAGTGCA | hsa-miR-152 | TCAGTGCATGACAGAACTTGG | 25 |
| hsa-miR-152 | CAGTGCA | hsa-miR-148a | TCAGTGCACTACAGAACTTTGT | 123 |
| | CAGTGCA | hsa-miR-148b | TCAGTGCATCACAGAACTTTGT | 93 |
| hsa-miR-181a | ACATTCA | hsa-miR-181b | AACATTCATTGCTGTCGGTGGGT | 67 |
| | ACATTCA | hsa-miR-181c | AACATTCAACCTGTCGGTGAGT | 124 |
| | ACATTCA | hsa-miR-181d | AACATTCATTGTTGTCGGTGGGT | 125 |
| hsa-miR-181b | ACATTCA | hsa-miR-181a | AACATTCAACGCTGTCGGTGAGT | 95 |
| | ACATTCA | hsa-miR-181c | AACATTCAACCTGTCGGTGAGT | 124 |
| | ACATTCA | hsa-miR-181d | AACATTCATTGTTGTCGGTGGGT | 125 |
| hsa-miR-192 | TGACCTA | hsa-miR-215 | ATGACCTATGAATTGACAGAC | 126 |
| hsa-miR-193a- | ACTGGCC | hsa-miR-193b | AACTGGCCCTCAAAGTCCCGCT | 71 |
| 3p | | | | |
| hsa-miR-193b | ACTGGCC | hsa-miR-193a-3p | AACTGGCCTACAAAGTCCCAGT | 218 |
| hsa-miR-196a | AGGTAGT | hsa-miR-196b | TAGGTAGTTTCCTGTTGTTGGG | 127 |
| hsa-miR-19b | GTGCAAA | hsa-miR-19a | TGTGCAAATCTATGCAAAACTGA | 128 |
| hsa-miR-200a | AACACTG | hsa-miR-141 | TAACACTGTCTGGTAAAGATGG | 69 |
| hsa-miR-200c | AATACTG | hsa-miR-200b | TAATACTGCCTGGTAATGATGA | 129 |
| | AATACTG | hsa-miR-429 | TAATACTGTCTGGTAAAACCGT | 130 |
| hsa-miR-21 | AGCTTAT | hsa-miR-590-5p | GAGCTTATTCATAAAAGTGCAG | 131 |
| hsa-miR-27b | TCACAGT | hsa-miR-27a | TTCACAGTGGCTAAGTTCCGC | 132 |
| hsa-miR-29a | AGGACCA | hsa-miR-29b | TAGCACCATTTGAAATCAGTGTT | 43 |
| | AGCACCA | hsa-miR-29c | TAGCACCATTTGAAATCGGTTA | 87 |
| hsa-miR-29b | AGCACCA | hsa-miR-29a | TAGCACCATCTGAAATCGGTTA | 51 |
| | AGCACCA | hsa-miR-29c | TAGCACCATTTGAAATCGGTTA | 87 |
| hsa-miR-29c | AGCACCA | hsa-miR-29a | TAGCACCATCTGAAATCGGTTA | 51 |
| | AGCACCA | hsa-miR-29b | TAGCACCATTTGAAATCAGTGTT | 43 |
| hsa-miR-34a | GGCAGTG | hsa-miR-34c-5p | AGGCAGTGTAGTTAGCTGATTGC | 133 |
| | GGCAGTG | hsa-miR-449a | TGGCAGTGTATTGTTAGCTGGT | 134 |
| | GGCAGTG | hsa-miR-449b | AGGCAGTGTATTGTTAGCTGGC | 135 |
| hsa-miR-363 | ATTGCAC | hsa-miR-25 | CATTGCACTTGTCTCGGTCTGA | 148 |
| | ATTGCAC | hsa-miR-32 | TATTGCACATTACTAAGTTGCA | 136 |
| | ATTGCAC | hsa-miR-367 | AATTGCACTTTAGCAATGGTGA | 137 |
| | ATTGCAC | hsa-miR-92a | TATTGCACTTGTCCCGGCCTGT | 13 |
| | ATTGCAC | hsa-miR-92b | TATTGCACTCGTCCCGGCCTCC | 19 |
| hsa-miR-372 | AAGTGCT | hsa-miR-302a | TAAGTGCTTCCATGTTTTGGTGA | 139 |
| | AAGTGCT | hsa-miR-302b | TAAGTGCTTCCATGTTTTAGTAG | 140 |
| | AAGTGCT | hsa-miR-302c | TAAGTGCTTCCATGTTTCAGTGG | 141 |
| | AAGTGCT | hsa-miR-302d | TAAGTGCTTCCATGTTTGAGTGT | 142 |
| | AAGTGCT | hsa-miR-373 | GAAGTGCTTCGATTTTGGGGTGT | 73 |
| | AAGTGCT | hsa-miR-520a-3p | AAAGTGCTTCCCTTTGGACTGT | 143 |
| | AAGTGCT | hsa-miR-520b | AAAGTGCTTCCTTTTAGAGGG | 144 |
| | AAGTGCT | hsa-miR-520c-3p | AAAGTGCTTCCTTTTAGAGGGT | 145 |
| | AAGTGCT | hsa-miR-520d-3p | AAAGTGCTTCTCTTTGGTGGGT | 146 |
| | AAGTGCT | hsa-miR-520e | AAAGTGCTTCCTTTTTGAGGG | 147 |
| hsa-miR-373 | AAGTGCT | hsa-miR-302a | TAAGTGCTTCCATGTTTTGGTGA | 139 |
| | AAGTGCT | hsa-miR-302b | TAAGTGCTTCCATGTTTTAGTAG | 140 |
| | AAGTGCT | hsa-miR-302c | TAAGTGCTTCCATGTTTCAGTGG | 141 |
| | AAGTGCT | hsa-miR-302d | TAAGTGCTTCCATGTTTGAGTGT | 142 |
| | AAGTGCT | hsa-miR-372 | AAAGTGCTGCGACATTTGAGCGT | 5 |
| | AAGTGCT | hsa-miR-520a-3p | AAAGTGCTTCCCTTTGGACTGT | 143 |
| | AAGTGCT | hsa-miR-520b | AAAGTGCTTCCTTTTAGAGGG | 144 |
| | AAGTGCT | hsa-miR-520c-3p | AAAGTGCTTCCTTTTAGAGGGT | 145 |
| | AAGTGCT | hsa-miR-520d-3p | AAAGTGCTTCTCTTTGGTGGGT | 146 |
| | AAGTGCT | hsa-miR-520e | AAAGTGCTTCCTTTTTGAGGG | 147 |
| hsa-miR-92a | ATTGCAC | hsa-miR-25 | CATTGCACTTGTCTCGGTCTGA | 148 |
| | ATTGCAC | hsa-miR-32 | TATTGCACATTACTAAGTTGCA | 136 |
| | ATTGCAC | hsa-miR-363 | AATTGCACGGTATCCATCTGTA | 59 |
| | ATTGCAC | hsa-miR-367 | AATTGCACTTTAGCAATGGTGA | 137 |
| | ATTGCAC | hsa-miR-92b | TATTGCACTCGTCCCGGCCTCC | 19 |
| hsa-miR-92b | ATTGCAC | hsa-miR-25 | CATTGCACTTGTCTCGGTCTGA | 148 |
| | ATTGCAC | hsa-miR-32 | TATTGCACATTACTAAGTTGCA | 136 |
| | ATTGCAC | hsa-miR-363 | AATTGCACGGTATCCATCTGTA | 59 |
| | ATTGCAC | hsa-miR-367 | AATTGCACTTTAGCAATGGTGA | 137 |
| | ATTGCAC | hsa-miR-92a | TATTGCACTTGTCCCGGCCTGT | 13 |
| hsa-miR-99a | ACCCGTA | hsa-miR-100 | AACCCGTAGATCCGAACTTGTG | 149 |
| | ACCCGTA | hsa-miR-99b | CACCCGTAGAACCGACCTTGCG | 150 |

For some of the microRNAs in Table 2, other microRNAs are known in the human genome that are located with close proximity on the genome (genomic cluster) (see Table 5) and may be similarly expressed together with the indicated miRs. These microRNAs from nearly the same genomic location may be substituted for the indicated miRs.

TABLE 5
microRNAs within the same genomic cluster (distance <10 kb)

| | | | | |
|---|---|---|---|---|
| | miRs within | | | |
| Indicated | the same | | Genomic | SEQ |
| miRs | genomic cluster | miR sequence | distance | ID# |

| | | | | |
|---|---|---|---|---|
| hsa-let-7e | hsa-miR-125a-3p | ACAGGTGAGGTTCTTGGGAGCC | 503 | 219 |
| | hsa-miR-125a-5p | TCCCTGAGACCCTTTAACCTGTGA | 503 | 220 |
| | hsa-miR-99b | CACCCGTAGAACCGACCTTGCG | 139 | 150 |
| | hsa-miR-99b* | CAAGCTCGTGTCTGTGGGTCCG | 139 | 151 |
| hsa-miR-106b | hsa-miR-25 | CATTGCACTTGTCTCGGTCTGA | 430 | 148 |
| | hsa-miR-25* | AGGCGGAGACTTGGGCAATTG | 430 | 152 |
| | hsa-miR-93 | CAAAGTGCTGTTCGTGCAGGTAG | 226 | 115 |
| | hsa-miR-93* | ACTGCTGAGCTAGCACTTCCCG | 226 | 153 |
| hsa-miR-141 | hsa-miR-200c | TAATACTGCCGGGTAATGATGGA | 405 | 3 |
| | hsa-miR-200c* | CGTCTTACCCAGCAGTGTTTGG | 405 | 154 |
| hsa-miR-145 | hsa-miR-143 | TGAGATGAAGCACTGTAGCTC | 1716 | 99 |
| | hsa-miR-143* | GGTGCAGTGCTGCATCTCTGGT | 1716 | 155 |
| hsa-miR-181a | hsa-miR-181b | AACATTCATTGCTGTCGGTGGGT | 178 | 67 |
| | hsa-miR-181b | AACATTCATTGCTGTCGGTGGGT | 1247 | 67 |
| hsa-miR-181b | hsa-miR-181a | AACATTCAACGCTGTCGGTGAGT | 178 | 95 |
| | hsa-miR-181a | AACATTCAACGCTGTCGGTGAGT | 1247 | 95 |
| | hsa-miR-181a* | ACCATCGACCGTTGATTGTACC | 178 | 156 |
| | hsa-miR-181a-2* | ACCACTGACCGTTGACTGTACC | 1247 | 157 |
| hsa-miR-182 | hsa-miR-183 | TATGGCACTGGTAGAATTCACT | 4523 | 158 |
| | hsa-miR-183* | GTGAATTACCGAAGGGCCATAA | 4523 | 159 |
| | hsa-miR-96 | TTTGGCACTAGCACATTTTTGCT | 4290 | 160 |
| | hsa-miR-96* | AATCATGTGCAGTGCCAATATG | 4290 | 161 |
| hsa-miR-192 | hsa-miR-194 | TGTAACAGCAACTCCATGTGGA | 208 | 37 |
| | hsa-miR-194* | CCAGTGGGGCTGCTGTTATCTG | 208 | 162 |
| hsa-miR-193b | hsa-miR-365 | TAATGCCCCTAAAAATCCTTAT | 5321 | 163 |
| hsa-miR-194 | hsa-miR-192 | CTGACCTATGAATTGACAGCC | 208 | 29 |
| | hsa-miR-192* | CTGCCAATTCCATAGGTCACAG | 208 | 164 |
| | hsa-miR-215 | ATGACCTATGAATTGACAGAC | 290 | 126 |
| hsa-miR-19b | hsa-miR-106a | AAAAGTGCTTACAGTGCAGGTAG | 519 | 165 |
| | hsa-miR-106a* | CTGCAATGTAAGCACTTCTTAC | 519 | 166 |
| | hsa-miR-17 | CAAAGTGCTTACAGTGCAGGTAG | 581 | 110 |
| | hsa-miR-17* | ACTGCAGTGAAGGCACTTGTAG | 581 | 167 |
| | hsa-miR-18a | TAAGGTGCATCTAGTGCAGATAG | 434 | 168 |
| | hsa-miR-18a* | ACTGCCCTAAGTGCTCCTTCTGG | 434 | 169 |
| | hsa-miR-18b | TAAGGTGCATCTAGTGCAGTTAG | 364 | 170 |
| | hsa-miR-18b* | TGCCCTAAATGCCCCTTCTGGC | 364 | 171 |
| | hsa-miR-19a | TGTGCAAATCTATGCAAAACTGA | 295 | 128 |
| | hsa-miR-19a* | AGTTTTGCATAGTTGCACTACA | 295 | 172 |
| | hsa-miR-20a | TAAAGTGCTTATAGTGCAGGTAG | 138 | 111 |
| | hsa-miR-20a* | ACTGCATTATGAGCACTTAAAG | 138 | 216 |
| | hsa-miR-20b | CAAAGTGCTCATAGTGCAGGTAG | 119 | 112 |
| | hsa-miR-20b* | ACTGTAGTATGGGCACTTCCAG | 119 | 173 |
| | hsa-miR-363 | AATTGCACGGTATCCATCTGTA | 307 | 59 |
| | hsa-miR-363* | CGGGTGGATCACGATGCAATTT | 307 | 174 |
| | hsa-miR-92a | TATTGCACTTGTCCCGGCCTGT | 136 | 13 |
| | hsa-miR-92a | TATTGCACTTGTCCCGGCCTGT | 144 | 13 |
| | hsa-miR-92a-1* | AGGTTGGGATCGGTTGCAATGCT | 136 | 175 |
| | hsa-miR-92a-2* | GGGTGGGGATTTGTTGCATTAC | 144 | 176 |
| hsa-miR-200a | hsa-miR-200b | TAATACTGCCTGGTAATGATGA | 768 | 129 |
| | hsa-miR-200b* | CATCTTACTGGGCAGCATTGGA | 768 | 177 |
| | hsa-miR-429 | TAATACTGTCTGGTAAAACCGT | 1138 | 130 |
| hsa-miR-200c | hsa-miR-141 | TAACACTGTCTGGTAAAGATGG | 405 | 69 |
| | hsa-miR-141* | CATCTTCCAGTACAGTGTTGGA | 405 | 178 |
| hsa-miR-214 | hsa-miR-199a-3p | ACAGTAGTCTGCACATTGGTTA | 5747 | 179 |
| | hsa-miR-199a-5p | CCCAGTGTTCAGACTACCTGTTC | 5747 | 180 |
| hsa-miR-27b | hsa-miR-23b | ATCACATTGCCAGGGATTACC | 270 | 181 |
| | hsa-miR-23b* | TGGGTTCCTGGCATGCTGATTT | 270 | 182 |
| | hsa-miR-24 | TGGCTCAGTTCAGCAGGAACAG | 576 | 183 |
| | hsa-miR-24-1* | TGCCTACTGAGCTGATATCAGT | 576 | 184 |
| hsa-miR-29a | hsa-miR-29b | TAGCACCATTTGAAATCAGTGTT | 732 | 43 |
| | hsa-miR-29b-1* | GCTGGTTTCATATGGTGGTTTAGA | 732 | 185 |
| hsa-miR-29b | hsa-miR-29a | TAGCACCATCTGAAATCGGTTA | 732 | 51 |
| | hsa-miR-29a* | ACTGATTTCTTTTGGTGTTCAG | 732 | 186 |
| | hsa-miR-29c | TAGCACCATTTGAAATCGGTTA | 586 | 87 |
| | hsa-miR-29c* | TGACCGATTTCTCCTGGTGTTC | 586 | 187 |
| hsa-miR-29c | hsa-miR-29b | TAGCACCATTTGAAATCAGTGTT | 586 | 43 |
| | hsa-miR-29b-2* | CTGGTTTCACATGGTGGCTTAG | 586 | 188 |
| hsa-miR-34b | hsa-miR-34c-3p | AATCACTAACCACACGGCCAGG | 511 | 189 |
| | hsa-miR-34c-5p | AGGCAGTGTAGTTAGCTGATTGC | 511 | 133 |
| hsa-miR-363 | hsa-miR-106a | AAAAGTGCTTACAGTGCAGGTAG | 826 | 165 |
| | hsa-miR-106a* | CTGCAATGTAAGCACTTCTTAC | 826 | 166 |
| | hsa-miR-18b | TAAGGTGCATCTAGTGCAGTTAG | 671 | 170 |
| | hsa-miR-18b* | TGCCCTAAATGCCCCTTCTGGC | 671 | 171 |
| | hsa-miR-19b | TGTGCAAATCCATGCAAAACTGA | 307 | 55 |
| | hsa-miR-19b-2* | AGTTTTGCAGGTTTGCATTTCA | 307 | 190 |
| | hsa-miR-20b | CAAAGTGCTCATAGTGCAGGTAG | 426 | 112 |
| | hsa-miR-20b* | ACTGTAGTATGGGCACTTCCAG | 426 | 173 |
| | hsa-miR-92a | TATTGCACTTGTCCCGGCCTGT | 163 | 13 |
| | hsa-miR-92a-2* | GGGTGGGGATTTGTTGCATTAC | 163 | 176 |
| hsa-miR-372 | hsa-miR-371-3p | AAGTGCCGCCATCTTTTGAGTGT | 217 | 191 |
| | hsa-miR-371-5p | ACTCAAACTGTGGGGGCACT | 217 | 192 |
| | hsa-miR-373 | GAAGTGCTTCGATTTTGGGGTGT | 803 | 73 |
| | hsa-miR-373* | ACTCAAAATGGGGGCGCTTTCC | 803 | 193 |
| hsa-miR-373 | hsa-miR-371-3p | AAGTGCCGCCATCTTTTGAGTGT | 1020 | 191 |
| | hsa-miR-371-5p | ACTCAAACTGTGGGGGCACT | 1020 | 192 |
| | hsa-miR-372 | AAAGTGCTGCGACATTTGAGCGT | 803 | 5 |
| hsa-miR-382 | hsa-miR-134 | TGTGACTGGTTGACCAGAGGGG | 381 | 194 |
| | hsa-miR-154 | TAGGTTATCCGTGTTGCCTTCG | 5453 | 195 |
| | hsa-miR-154* | AATCATACACGGTTGACCTATT | 5453 | 196 |
| | hsa-miR-377 | ATCACACAAAGGCAACTTTTGT | 7738 | 197 |
| | hsa-miR-377* | AGAGGTTGCCCTTGGTGAATTC | 7738 | 198 |
| | hsa-miR-381 | TATACAAGGGCAAGCTCTCTGT | 8404 | 199 |
| | hsa-miR-453 | AGGTTGTCCGTGGTGAGTTCGCA | 1888 | 200 |
| | hsa-miR-485-3p | GTCATACACGGCTCTCCTCTCT | 1112 | 201 |
| | hsa-miR-485-5p | AGAGGCTGGCCGTGATGAATTC | 1112 | 202 |
| | hsa-miR-487a | AATCATACAGGGACATCCAGTT | 1864 | 203 |
| | hsa-miR-487b | AATCGTACAGGGTCATCCACTT | 7858 | 204 |
| | hsa-miR-496 | TGAGTATTACATGGCCAATCTC | 6270 | 205 |
| | hsa-miR-539 | GGAGAAATTATCCTTGGTGTGT | 6986 | 206 |
| | hsa-miR-544 | ATTCTGCATTTTTAGCAAGTTC | 5645 | 207 |
| | hsa-miR-655 | ATAATACATGGTTAACCTCTTT | 4742 | 208 |
| | hsa-miR-668 | TGTCACTCGGCTCGGCCCACTAC | 955 | 209 |
| | hsa-miR-889 | TTAATATCGGACAACCATTGT | 6406 | 210 |
| hsa-miR-509-3p | hsa-miR-509-3-5p | TACTGCAGACGTGGCAATCATG | 883 | 211 |
| | hsa-miR-509-3-5p | TACTGCAGACGTGGCAATCATG | 888 | 211 |
| | hsa-miR-509-3p | TGATTGGTACGTCTGTGGGTAG | 883 | 212 |
| | hsa-miR-509-3p | TGATTGGTACGTCTGTGGGTAG | 888 | 212 |
| | hsa-miR-509-3p | TGATTGGTACGTCTGTGGGTAG | 1771 | 212 |
| | hsa-miR-509-5p | TACTGCAGACAGTGGCAATCA | 883 | 213 |
| | hsa-miR-509-5p | TACTGCAGACAGTGGCAATCA | 888 | 213 |
| | hsa-miR-509-5p | TACTGCAGACAGTGGCAATCA | 1771 | 213 |
| hsa-miR-92a | hsa-miR-106a | AAAAGTGCTTACAGTGCAGGTAG | 663 | 165 |
| | hsa-miR-106a* | CTGCAATGTAAGCACTTCTTAC | 663 | 166 |
| | hsa-miR-17 | CAAAGTGCTTACAGTGCAGGTAG | 717 | 110 |
| | hsa-miR-17* | ACTGCAGTGAAGGCACTTGTAG | 717 | 167 |
| | hsa-miR-18a | TAAGGTGCATCTAGTGCAGATAG | 570 | 168 |
| | hsa-miR-18a* | ACTGCCCTAAGTGCTCCTTCTGG | 570 | 169 |
| | hsa-miR-18b | TAAGGTGCATCTAGTGCAGTTAG | 508 | 170 |
| | hsa-miR-18b* | TGCCCTAAATGCCCCTTCTGGC | 508 | 171 |
| | hsa-miR-19a | TGTGCAAATCTATGCAAAACTGA | 431 | 128 |
| | hsa-miR-19a* | AGTTTTGCATAGTTGCACTACA | 431 | 172 |
| | hsa-miR-19b | TGTGCAAATCCATGCAAAACTGA | 136 | 55 |
| | hsa-miR-19b | TGTGCAAATCCATGCAAAACTGA | 144 | 55 |
| | hsa-miR-19b-1* | AGTTTTGCAGGTTTGCATCCAGC | 136 | 215 |
| | hsa-miR-19b-2* | AGTTTTGCAGGTTTGCATTTCA | 144 | 190 |
| | hsa-miR-20a | TAAAGTGCTTATAGTGCAGGTAG | 274 | 111 |
| | hsa-miR-20a* | ACTGCATTATGAGCACTTAAAG | 274 | 216 |
| | hsa-miR-20b | CAAAGTGCTCATAGTGCAGGTAG | 263 | 112 |
| | hsa-miR-20b* | ACTGTAGTATGGGCACTTCCAG | 263 | 173 |
| | hsa-miR-363 | AATTGCACGGTATCCATCTGTA | 163 | 59 |
| | hsa-miR-363* | CGGGTGGATCACGATGCAATTT | 163 | 174 |
| hsa-miR-99a | hsa-let-7c | TGAGGTAGTAGGTTGTATGGTT | 710 | 105 |
| | hsa-let-7c* | TAGAGTTACACCCTGGGAGTTA | 710 | 217 |

For some of the microRNAs in Table 2, other microRNAs are known in the human genome that have similar sequence (less than 6 mismatches in the sequence) (see Table 6), and therefore may be also captured by probes with the same design. These microRNAs with similar overall sequence may be substituted for the indicated miRs.

TABLE 6
microRNAs with similar sequence

| | | | | |
|---|---|---|---|---|
| | miRs in | | | |
| | sequence | Cluster | | SEQ |
| Indicated miRs | cluster | ID | Sequence | ID# |

| | | | | |
|---|---|---|---|---|
| hsa-miR-148b | hsa-miR-148a | 1 | TCAGTGCACTACAGAACTTTGT | 123 |
| | hsa-miR-152 | 1 | TCAGTGCATGACAGAACTTGG | 25 |
| hsa-miR-152 | hsa-miR-148a | 1 | TCAGTGCACTACAGAACTTTGT | 123 |
| | hsa-miR-148b | 1 | TCAGTGCATCACAGAACTTTGT | 93 |
| hsa-miR-92a | hsa-miR-92b | 10 | TATTGCACTCGTCCCGGCCTCC | 19 |
| hsa-miR-92b | hsa-miR-92a | 10 | TATTGCACTTGTCCCGGCCTGT | 13 |
| hsa-miR-19b | hsa-miR-19a | 15 | TGTGCAAATCTATGCAAAACTGA | 128 |
| hsa-miR-141 | hsa-miR-200a | 22 | TAACACTGTCTGGTAACGATGT | 200a |
| hsa-miR-200a | hsa-miR-141 | 22 | TAACACTGTCTGGTAAAGATGG | 69 |
| hsa-miR-130a | hsa-miR-130b | 30 | CAGTGCAATGATGAAAGGGCAT | 118 |
| hsa-miR-99a | hsa-miR-100 | 36 | AACCCGTAGATCCGAACTTGTG | 149 |
| | hsa-miR-99b | 36 | CACCCGTAGAACCGACCTTGCG | 150 |
| hsa-miR-27b | hsa-miR-27a | 37 | TTCACAGTGGCTAAGTTCCGC | 132 |
| hsa-let-7e | hsa-let-7a | 4 | TGAGGTAGTAGGTTGTATAGTT | 103 |
| | hsa-let-7b | 4 | TGAGGTAGTAGGTTGTGTGGTT | 104 |
| | hsa-let-7c | 4 | TGAGGTAGTAGGTTGTATGGTT | 105 |
| | hsa-let-7d | 4 | AGAGGTAGTAGGTTGCATAGTT | 106 |
| | hsa-let-7f | 4 | TGAGGTAGTAGATTGTATAGTT | 107 |
| | hsa-let-7g | 4 | TGAGGTAGTAGTTTGTACAGTT | 108 |
| | hsa-miR-98 | 4 | TGAGGTAGTAAGTTGTATTGTT | 109 |
| hsa-miR-196a | hsa-miR-196b | 51 | TAGGTAGTTTCCTGTTGTTGGG | 127 |
| hsa-miR-29a | hsa-miR-29b | 56 | TAGCACCATTTGAAATCAGTGTT | 43 |
| | hsa-miR-29c | 56 | TAGCACCATTTGAAATCGGTTA | 87 |
| hsa-miR-29b | hsa-miR-29a | 56 | TAGCACCATCTGAAATCGGTTA | 151 |
| | hsa-miR-29c | 56 | TAGCACCATTTGAAATCGGTTA | 87 |
| hsa-miR-29c | hsa-miR-29a | 56 | TAGCACCATCTGAAATCGGTTA | 51 |
| | hsa-miR-29b | 56 | TAGCACCATTTGAAATCAGTGTT | 43 |
| hsa-miR-200c | hsa-miR-200b | 60 | TAATACTGCCTGGTAATGATGA | 129 |
| hsa-miR-193a-3p | hsa-miR-193b | 62 | AACTGGCCCTCAAAGTCCCGCT | 71 |
| hsa-miR-193b | hsa-miR-193a-3p | 62 | AACTGGCCTACAAAGTCCCAGT | 218 |
| hsa-miR-182 | hsa-miR-183 | 63 | TATGGCACTGGTAGAATTCACT | 158 |
| hsa-miR106b | hsa-miR-106a | 64 | AAAAGTGCTTACAGTGCAGGTAG | 165 |
| | hsa-miR-17 | 64 | CAAAGTGCTTACAGTGCAGGTAG | 110 |
| | hsa-miR-20a | 64 | TAAAGTGCTTATAGTGCAGGTAG | 111 |
| | hsa-miR-20b | 64 | CAAAGTGCTCATAGTGCAGGTAG | 112 |
| | hsa-miR-93 | 64 | CAAAGTGCTGTTCGTGCAGGTAG | 115 |
| hsa-miR-181a | hsa-miR-181b | 66 | AACATTCATTGCTGTCGGTGGGT | 67 |
| | hsa-miR-181c | 66 | AACATTCAACCTGTCGGTGAGT | 124 |
| | hsa-miR-181d | 66 | AACATTCATTGTTGTCGGTGGGT | 125 |
| hsa-miR-181b | hsa-miR-181a | 66 | AACATTCAACGCTGTCGGTGAGT | 95 |
| | hsa-miR-181c | 66 | AACATTCAACCTGTCGGTGAGT | 124 |
| | hsa-miR-181d | 66 | AACATTCATTGTTGTCGGTGGGT | 125 |
| hsa-miR-146a | hsa-miR-146b-5p | 67 | TGAGAACTGAATTCCATAGGCT | 122 |
| hsa-miR-10b | hsa-miR-10a | 7 | TACCCTGTAGATCCGAATTTGTG | 116 |
| hsa-miR-192 | hsa-miR-215 | 72 | ATGACCTATGAATTGACAGAC | 126 |

### REFERENCES

1. Bentwich, I. et al. Identification of hundreds of conserved and nonconserved human microRNAs. Nat Genet (2005).
2. Farh, K. K. et al. The Widespread Impact of Mammalian MicroRNAs on mRNA Repression and Evolution. Science (2005).
3. Griffiths-Jones, S., Grocock, R. J., van Dongen, S., Bateman, A. & Enright, A. J. miRBase: microRNA sequences, targets and gene nomenclature. Nucleic Acids Res 34, D140-4 (2006).
4. He, L. et al. A microRNA polycistron as a potential human oncogene. Nature 435, 828-33 (2005).
5. Baskerville, S. & Bartel, D. P. Microarray profiling of microRNAs reveals frequent coexpression with neighboring miRNAs and host genes. Rna 11, 241-7 (2005).
6. Landgraf, P. et al. A Mammalian microRNA Expression Atlas Based on Small RNA Library Sequencing. Cell 129, 1401-14 (2007).
7. Volinia, S. et al. A microRNA expression signature of human solid tumors defines cancer gene targets. Proc Natl Acad Sci USA (2006).
8. Lu, J. et al. MicroRNA expression profiles classify human cancers. Nature 435, 834-8 (2005).
9. Varadhachary, G. R., Abbruzzese, J. L. & Lenzi, R. Diagnostic strategies for unknown primary cancer. Cancer 100, 1776-85 (2004).
10. Pimiento, J. M., Teso, D., Malkan, A., Dudrick, S. J. & Palesty, J. A. Cancer of unknown primary origin: a decade of experience in a community-based hospital. Am J Surg 194, 833-7; discussion 837-8 (2007).
11. Shaw, P. H., Adams, R., Jordan, C. & Crosby, T. D. A clinical review of the investigation and management of carcinoma of unknown primary in a single cancer network. Clin Oncol (R Coll Radiol) 19, 87-95 (2007).
12. Hainsworth, J. D. & Greco, F. A. Treatment of patients with cancer of an unknown primary site. N Engl J Med 329, 257-63 (1993).
13. Blaszyk, H., Hartmann, A. & Bjornsson, J. Cancer of unknown primary: clinicopathologic correlations. Apmis 111, 1089-94 (2003).
14. Bloom, G. et al. Multi-platform, multi-site, microarray-based human tumor classification. Am J Pathol 164, 9-16 (2004).
15. Ma, X. J. et al. Molecular classification of human cancers using a 92-gene real-time quantitative polymerase chain reaction assay. Arch Pathol Lab Med 130, 465-73 (2006).
16. Talantov, D. et al. A quantitative reverse transcriptase-polymerase chain reaction assay to identify metastatic carcinoma tissue of origin. J Mol Diagn 8, 320-9 (2006).
17. Tothill, R. W. et al. An expression-based site of origin diagnostic method designed for clinical application to cancer of unknown origin. Cancer Res 65, 4031-40 (2005).
18. Shedden, K. A. et al. Accurate molecular classification of human cancers based on gene expression using a simple classifier with a pathological tree-based framework. Am J Pathol 163, 1985-95 (2003).
19. Raver-Shapira, N. et al. Transcriptional Activation of miR-34a Contributes to p53-Mediated Apoptosis. Mol Cell (2007).
20. Xiao, C. et al. MiR-150 Controls B Cell Differentiation by Targeting the Transcription Factor c-Myb. Cell 131, 146-59 (2007).

The foregoing description of the specific embodiments so fully reveals the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It should be understood that the detailed description and specific examples, while indicating preferred embodiments of the invention, are given by way of illustration only, since various changes and modifications within the spirit and scope of the invention will become apparent to those skilled in the art from this detailed description.

1. A method of classifying a tissue of origin of a biological sample, the method comprising:
(a) obtaining a biological sample from a subject;
(b) determining an expression profile in said sample of nucleic acid sequences selected from the group consisting of SEQ ID NOS: 1-96, or a sequence having at least about 80% identity thereto; and
(c) comparing said expression profile to a reference expression profile;
whereby the differential expression of any of said nucleic acid sequences allows the classification of the tissue of origin of said sample.

2. The method of claim 1, wherein said tissue is selected from the group consisting of liver, lung, bladder, prostate, breast, colon, ovary, testis, stomach, thyroid, pancreas, brain, endometrium, head and neck, lymph node, kidney, melanocytes, meninges, thymus and prostate.

3. A method of classifying a cancer or hyperplasia, said method comprising:
(a) obtaining a biological sample from a subject;
(b) measuring the relative abundance in said sample of nucleic acid sequences selected from the group consisting of SEQ ID NOS: 1-96 or a sequence having at least about 80% identity thereto; and
(c) comparing said obtained measurement to a reference abundance of said nucleic acid;
whereby the differential expression of any of said nucleic acid sequences allows the classification of said cancer or hyperplasia.

4. The method of claim 3, wherein said sample is obtained from a subject with cancer of unknown primary (CUP), with a primary cancer or with a metastatic cancer.

5. The method of claim 3, wherein said cancer is selected from the group consisting of liver cancer, lung cancer, bladder cancer, prostate cancer, breast cancer, colon cancer, ovarian cancer, testicular cancer, stomach cancer, thyroid cancer, pancreas cancer, brain cancer, endometrium cancer, head and neck cancer, lymph node cancer, kidney cancer, melanoma, meninges cancer, thymus cancer, prostate cancer, gastrointestinal stromal cancer and sarcoma.

6-20. (canceled)

21. The method of claim 1, wherein said biological sample is selected from the group consisting of bodily fluid, a cell line and a tissue sample.

22. The method of claim 21, wherein said tissue is a fresh, frozen, fixed, wax-embedded or formalin fixed paraffin-embedded (FFPE) tissue.

23. The method of claim 1, wherein said expression profile is a transcriptional profile.

24. The method of claim 1, wherein said method further comprises use of at least one classifier algorithm.

25. The method of claim 24, wherein said at least one classifier is selected from the group consisting of decision tree classifier, logistic regression classifier, nearest neighbor classifier, neural network classifier, Gaussian mixture model (GMM) and Support Vector Machine (SVM) classifier.

26-50. (canceled)

51. A method of classifying a tissue of origin of a biological sample, the method comprising:
(a) obtaining a biological sample from a subject;
(b) determining an individual gene expression of each gene in a gene set of said sample, wherein said gene set comprises microRNAs; and
(c) classifying the tissue of origin for said sample by at least one classifier.

52. The method of claim 51, wherein the at least one classifier is a decision tree model.

53. A kit for cancer classification, said kit comprising a probe comprising a nucleic acid sequence selected from the group consisting of:
(a) SEQ ID NOS: 1-96;
(b) complementary sequence of (a); and
(c) a sequence having at least about 80% identity to (a) or (b).

54. The method of claim 5, wherein said specific cancers are further selected from the group consisting of:
a) for liver cancer, the type of liver cancer is selected from the group consisting of liver hepatoma, liver hepatocellular carcinoma (HCC), liver cholangiocarcinoma, liver hepatoblastoma, liver angiosarcoma, liver hepatocellular adenoma, and liver hemangioma,
b) for pancreas cancer, the type of pancreas cancer is selected from the group consisting of pancreas ductal adenocarcinoma, pancreas insulinoma, pancreas glucagonoma, pancreas gastrinoma, pancreas carcinoid tumors, and pancreas vipoma,
c) for bladder cancer, the type of bladder cancer is selected from the group consisting of bladder squamous cell carcinoma, bladder transitional cell carcinoma and bladder adenocarcinoma,
d) for prostate cancer, the type of prostate cancer is selected from the group consisting of prostate adenocarcinoma, prostate sarcoma and benign prostatic hyperplasia (BPH),
e) for testis cancer, the type of testis cancer is selected from the group consisting of seminoma, testis teratoma, testis embryonal carcinoma, testis teratocarcinoma, testis choriocarcinoma, testis sarcoma, testis interstitial cell carcinoma, testis fibroma, testis fibroadenoma, testis adenomatoid tumors and testis lipoma,
f) for lung cancer, the type of lung cancer is selected from the group consisting of lung carcinoid, lung pleural mesothelioma and lung squamous cell carcinoma,
g) for ovarian cancer, the type of ovarian cancer is selected from the group consisting of ovarian carcinoma, unclassified ovarian carcinoma, serous papillary carcinoma, ovarian granulosa-thecal cell tumors, ovarian dysgerminoma and ovarian malignant teratoma,
h) for gastrointestinal stromal cancer, the type of gastrointestinal stromal cancer is selected from the group consisting of small intestine adenocarcinoma and small intestine carcinoid tumor,
i) for brain cancer the type of brain cancer is selected from the group consisting of glioblastoma, glioma, meningioma, astrocytoma, medulloblastoma, oligodendroglioma, neuroectodermal cancer and neuroblastoma,
j) for breast cancer, the type of breast cancer is selected from the group consisting of lobular carcinoma and ductal carcinoma,
k) for head and neck cancer, the type of head and neck cancer is squamous cell carcinoma,
l) for colon cancer, the type of colon cancer is adenocarcinoma,
m) for endometrium cancer, the type of endometrium cancer is endometrial adenocarcinoma,
n) for lymph node cancer, the type of lymph node cancer is Hodgkin's lymphoma, and
o) for thyroid cancer, the type of thyroid cancer is papillary carcinoma.

55. The method of claim 3 for classifying a cancer of the following origins, the method comprising measuring the relative abundance of the provided nucleic acid sequence or a sequence having at least about 80% identity thereto in said sample:
a) for classifying liver cancer, the nucleic acid sequence selected from the group consisting of SEQ ID NOS: 1-4,
b) for classifying a cancer of testicular origin, the nucleic acid sequence selected from the group consisting of SEQ ID NOS: 1-6,
c) for classifying a cancer of lung origin, the nucleic acid sequence selected from the group consisting of SEQ ID NOS: 1-8, 25, 26, 33, 34, 37, 38, 45, 46, 49, 50, 57-64, 69-84, 95 and 96,
d) for classifying a cancer of lung carcinoid origin, the nucleic acid sequence selected from the group consisting of SEQ ID NOS: 1-8, 31, 32, 37, 38, 45-48, 95 and 96,
e) for classifying a cancer of lung pleura origin, the nucleic acid sequence selected from the group consisting of SEQ ID NOS: 1-14, 19-40, 95 and 96,
f) for classifying a cancer of lung squamous origin, the nucleic acid sequence selected from the group consisting of SEQ ID NOS: 1-8, 29, 30, 33, 34, 37, 38, 45, 46, 57-64, 69-74, 85, 86 and 89-96,
g) for classifying a cancer of pancreatic origin, the nucleic acid sequence selected from the group consisting of SEQ ID NOS: 1-8, 31, 32, 37, 38, 45-56, 95 and 96,
h) for classifying a cancer of colon origin, the nucleic acid sequence selected from the group consisting of SEQ ID NOS: 1-8, 31, 32, 37, 38, 45-52, 95 and 96,
i) for classifying a cancer of head and neck origin, the nucleic acid sequence selected from the group consisting of SEQ ID NOS: 1-8, 29, 30, 33, 34, 37, 38, 45, 46, 57-64, 69-74, 85, 86 and 89-96,
j) for classifying a cancer of ovarian origin, the nucleic acid sequence selected from the group consisting of SEQ ID NOS: 1-8, 33, 34, 37, 38, 45, 46, 49, 50, 57-64, 69-90, 95 and 96,
k) for classifying a cancer of gastrointestinal stromal origin, the nucleic acid sequence selected from the group consisting of SEQ ID NOS: 1-14, 19-36, 41-44, 95 and 96,
l) for classifying a cancer of brain origin, the nucleic acid sequence selected from the group consisting of SEQ ID NOS: 1-14, 19-24, 95 and 96,
m) for classifying a cancer of breast origin, the nucleic acid sequence selected from the group consisting of SEQ ID NOS: 1-8, 33, 34, 37, 38, 45, 46, 49, 50, 57-68, 95 and 96,
n) for classifying a cancer of bladder origin, the nucleic acid sequence selected from the group consisting of SEQ ID NOS: 1-8, 25, 26, 33, 34, 37, 38, 45, 46, 49, 50, 57-64, 69-84, 95 and 96,
o) for classifying a cancer of prostate origin, the nucleic acid sequence selected from the group consisting of SEQ ID NOS: 1-8, 33, 34, 37, 38, 45, 46, 49, 50, 57-68, 95 and 96,
p) for classifying a cancer of thyroid origin, the nucleic acid sequence selected from the group consisting of SEQ ID NOS: 1-8, 33, 34, 37, 38, 45, 46, 49, 50, 57-64, 69-78, 95 and 96,
q) for classifying a cancer of endometrium origin, the nucleic acid sequence selected from the group consisting of SEQ ID NOS: 1-8, 33, 34, 37, 38, 45, 46, 49, 50, 57-64, 69-90, 95 and 96,
r) for classifying a cancer of kidney origin, the nucleic acid sequence selected from the group consisting of SEQ ID NOS: 1-14, 19-40, 95 and 96,
s) for classifying a cancer of melanocyte origin, the nucleic acid sequence selected from the group consisting of SEQ ID NOS: 1-18, 95 and 96,
t) for classifying a cancer of meninges origin, the nucleic acid sequence selected from the group consisting of SEQ ID NOS: 1-14, 19-28, 95 and 96,
u) for classifying a cancer of sarcoma origin, the nucleic acid sequence selected from the group consisting of SEQ ID NOS: 1-14, 19-36, 41-44, 95 and 96,
v) for classifying a cancer of stomach origin, the nucleic acid sequence selected from the group consisting of SEQ ID NOS: 1-8, 31, 32, 37, 38, 45-56, 95 and 96,
w) for classifying a cancer of lymph node origin, the nucleic acid sequence selected from the group consisting of SEQ ID NOS: 1-18, 95 and 96,
x) for classifying a cancer of thymus-B2 origin, the nucleic acid sequence selected from the group consisting of SEQ ID NOS: 1-14, 19-28, 95 and 96, and
y) for classifying a cancer of thymus-B3 origin, the nucleic acid sequence selected from the group consisting of SEQ ID NOS: 1-8, 29, 30, 33, 34, 37, 38, 45, 46, 49, 50, 57-64, 69-78, 95 and 96,
wherein the abundance of said nucleic acid sequence is indicative of a cancer of the provided origins.

56. The method of claim 3, wherein said biological sample is selected from the group consisting of bodily fluid, a cell line and a tissue sample.

57. The method of claim 3, wherein said method further comprises use of at least one classifier algorithm.


SEQUENCE LISTING (220 sequences)

SEQ ID NO: 1 - RNA, 23 nt, Homo Sapiense
uggagugugacaaugguguuugu

SEQ ID NO: 2 - RNA, 85 nt, Homo Sapiense
ccuuagcagagcuguggagugugacaaugguguuugugucuaaacuaucaaacgccauuaucacacuaaauagcuacugcuaggc

SEQ ID NO: 3 - RNA, 22 nt, Homo Sapiense
uaauacugccggguaaugaugg

SEQ ID NO: 4 - RNA, 68 nt, Homo Sapiense
cccucgucuuacccagcaguguuugggugcgguugggagucucuaauacugccggguaaugauggagg

SEQ ID NO: 5 - RNA, 23 nt, Homo Sapiense
aaagugcugcgacauuugagcgu

SEQ ID NO: 6 - RNA, 67 nt, Homo Sapiense
gugggccucaaauguggagcacuauucugauguccaaguggaaagugcugcgacauuugagcgucac

SEQ ID NO: 7 - RNA, 22 nt, Homo Sapiense
uccuucauuccaccggagucug

SEQ ID NO: 8 - RNA, 110 nt, Homo Sapiense
aaagauccucagacaauccaugugcuucucuuguccuucauuccaccggagucugucucauacccaaccagauuucaguggagugaaguucaggaggcauggagcugaca

SEQ ID NO: 9 - RNA, 22 nt, Homo Sapiense
ugagaacugaauuccauggguu

SEQ ID NO: 10 - RNA, 99 nt, Homo Sapiense
ccgauguguauccucagcuuugagaacugaauuccauggguugugucagugucagaccucugaaauucaguucuucagcugggauaucucugucaucgu

SEQ ID NO: 11 - RNA, 22 nt, Homo Sapiense
uaacacugucugguaacgaugu

SEQ ID NO: 12 - RNA, 90 nt, Homo Sapiense
ccgggccccugugagcaucuuaccggacagugcuggauuucccagcuugacucuaacacugucugguaacgauguucaaaggugacccgc

SEQ ID NO: 13 - RNA, 22 nt, Homo Sapiense
uauugcacuugucccggccugu

SEQ ID NO: 14 - RNA, 78 nt, Homo Sapiense
cuuucuacacagguugggaucgguugcaaugcuguguuucuguaugguauugcacuugucccggccuguugaguuugg

SEQ ID NO: 15 - RNA, 23 nt, Homo Sapiense
uguaguguuuccuacuuuaugga

SEQ ID NO: 16 - RNA, 87 nt, Homo Sapiense
gacagugcagucacccauaaaguagaaagcacuacuaacagcacuggaggguguaguguuuccuacuuuauggaugaguguacugug

SEQ ID NO: 17 - RNA, 23 nt, Homo Sapiense
ugauugguacgucuguggguaga

SEQ ID NO: 18 - RNA, 94 nt, Homo Sapiense
caugcugugugugguacccuacugcagacaguggcaaucauguauaauuaaaaaugauugguacgucuguggguagaguacugcaugacacaug

SEQ ID NO: 19 - RNA, 21 nt, Homo Sapiense
uauugcacucgucccggccuc

SEQ ID NO: 20 - RNA, 96 nt, Homo Sapiense
cgggccccgggcgggcgggagggacgggacgcggugcaguguuguuuuuucccccgccaauauugcacucgucccggccuccggcccccccggccc

SEQ ID NO: 21 - RNA, 21 nt, Homo Sapiense
uaaagcuagauaaccgaaagu

SEQ ID NO: 22 - RNA, 89 nt, Homo Sapiense
cgggguugguuguuaucuuugguuaucuagcuguaugagugguguggagucuucauaaagcuagauaaccgaaaguaaaaauaacccca

SEQ ID NO: 23 - RNA, 22 nt, Homo Sapiense
uuaaggcacgcggugaaugcca

SEQ ID NO: 24 - RNA, 109 nt, Homo Sapiense
aucaagauuagaggcucugcucuccguguucacagcggaccuugauuuaaugucauacaauuaaggcacgcggugaaugccaagagcggagccuacggcugcacuugaa

SEQ ID NO: 25 - RNA, 22 nt, Homo Sapiense
ucagugcaugacagaacuuggg

SEQ ID NO: 26 - RNA, 87 nt, Homo Sapiense
ugucccccccggcccagguucugugauacacuccgacucgggcucuggagcagucagugcaugacagaacuugggcccggaaggacc

SEQ ID NO: 27 - RNA, 22 nt, Homo Sapiense
cagugcaauguuaaaagggcau

SEQ ID NO: 28 - RNA, 89 nt, Homo Sapiense
ugcugcuggccagagcucuuuucacauugugcuacugucugcaccugucacuagcagugcaauguuaaaagggcauuggccguguagug

SEQ ID NO: 29 - RNA, 21 nt, Homo Sapiense
cugaccuaugaauugacagcc

SEQ ID NO: 30 - RNA, 110 nt, Homo Sapiense
gccgagaccgagugcacagggcucugaccuaugaauugacagccagugcucucgucuccccucuggcugccaauuccauaggucacagguauguucgccucaaugccagc

SEQ ID NO: 31 - RNA, 22 nt, Homo Sapiense
uagcuuaucagacugauguuga

SEQ ID NO: 32 - RNA, 72 nt, Homo Sapiense
ugucggguagcuuaucagacugauguugacuguugaaucucauggcaacaccagucgaugggcugucugaca

SEQ ID NO: 33 - RNA, 22 nt, Homo Sapiense
cugugcgugugacagcggcuga

SEQ ID NO: 34 - RNA, 110 nt, Homo Sapiense
acccggcagugccuccaggcgcagggcagccccugcccaccgcacacugcgcugccccagacccacugugcgugugacagcggcugaucugugccugggcagcgcgaccc

SEQ ID NO: 35 - RNA, 23 nt, Homo Sapiense
uaggcagugucauuagcugauug

SEQ ID NO: 36 - RNA, 84 nt, Homo Sapiense
gugcucgguuuguaggcagugucauuagcugauuguacuguggugguuacaaucacuaacuccacugccaucaaaacaaggcac

SEQ ID NO: 37 - RNA, 22 nt, Homo Sapiense
uguaacagcaacuccaugugga

SEQ ID NO: 38 - RNA, 85 nt, Homo Sapiense
augguguuaucaaguguaacagcaacuccauguggacuguguaccaauuuccaguggagaugcuguuacuuuugaugguuaccaa

SEQ ID NO: 39 - RNA, 22 nt, Homo Sapiense
gaaguuguucgugguggauucg

SEQ ID NO: 40 - RNA, 76 nt, Homo Sapiense
uacuugaagagaaguuguucgugguggauucgcuuuacuuaugacgaaucauucacggacaacacuuuuuucagua

SEQ ID NO: 41 - RNA, 21 nt, Homo Sapiense
ucgugucuuguguugcagccg

SEQ ID NO: 42 - RNA, 109 nt, Homo Sapiense
ggucgggcucaccaugacacagugugagaccucgggcuacaacacaggacccgggcgcugcucugaccccucgugucuuguguugcagccggagggacgcagguccgca

SEQ ID NO: 43 - RNA, 23 nt, Homo Sapiense
uagcaccauuugaaaucaguguu

SEQ ID NO: 44 - RNA, 81 nt, Homo Sapiense
cuucaggaagcugguuucauauggugguuuagauuuaaauagugauugucuagcaccauuugaaaucaguguucuuggggg

SEQ ID NO: 45 - RNA, 24 nt, Homo Sapiense
guccaguuuucccaggaaucccuu

SEQ ID NO: 46 - RNA, 88 nt, Homo Sapiense
caccuuguccucacgguccaguuuucccaggaaucccuuagaugcuaagauggggauuccuggaaauacuguucuugaggucaugguu

SEQ ID NO: 47 - RNA, 21 nt, Homo Sapiense
ugagguaggagguuguauagu

SEQ ID NO: 48 - RNA, 79 nt, Homo Sapiense
cccgggcugagguaggagguuguauaguugaggaggacacccaaggagaucacuauacggccuccuagcuuuccccagg

SEQ ID NO: 49 - RNA, 21 nt, Homo Sapiense
ugagguaguaguuugugcugu

SEQ ID NO: 50 - RNA, 84 nt, Homo Sapiense
cuggcugagguaguaguuugugcuguuggucggguugugacauugcccgcuguggagauaacugcgcaagcuacugccuugcua

SEQ ID NO: 51 - RNA, 21 nt, Homo Sapiense
uagcaccaucugaaaucgguu

SEQ ID NO: 52 - RNA, 64 nt, Homo Sapiense
augacugauuucuuuugguguucagagucaauauaauuuucuagcaccaucugaaaucgguuau

SEQ ID NO: 53 - RNA, 21 nt, Homo Sapiense
acagcaggcacagacaggcag

SEQ ID NO: 54 - RNA, 110 nt, Homo Sapiense
ggccuggcuggacagaguugucaugugucugccugucuacacuugcugugcagaacauccgcucaccuguacagcaggcacagacaggcagucacaugacaacccagccu

SEQ ID NO: 55 - RNA, 23 nt, Homo Sapiense
ugugcaaauccaugcaaaacuga

SEQ ID NO: 56 - RNA, 87 nt, Homo Sapiense
cacuguucuaugguuaguuuugcagguuugcauccagcugugugauauucugcugugcaaauccaugcaaaacugacugugguagug

SEQ ID NO: 57 - RNA, 21 nt, Homo Sapiense
uagguaguuucauguuguugg

SEQ ID NO: 58 - RNA, 70 nt, Homo Sapiense
gugaauuagguaguuucauguuguugggccuggguuucugaacacaacaacauuaaaccacccgauucac

SEQ ID NO: 59 - RNA, 22 nt, Homo Sapiense
aauugcacgguauccaucugua

SEQ ID NO: 60 - RNA, 75 nt, Homo Sapiense
uguugucggguggaucacgaugcaauuuugaugaguaucauaggagaaaaauugcacgguauccaucuguaaacc

SEQ ID NO: 61 - RNA, 21 nt, Homo Sapiense
ggcaagaugcuggcauagcug

SEQ ID NO: 62 - RNA, 71 nt, Homo Sapiense
ggagaggaggcaagaugcuggcauagcuguugaacugggaaccugcuaugccaacauauugccaucuuucc

SEQ ID NO: 63 - RNA, 21 nt, Homo Sapiense
aacuggccuacaaagucccag

SEQ ID NO: 64 - RNA, 88 nt, Homo Sapiense
cgaggaugggagcugagggcugggucuuugcgggcgagaugagggugucggaucaacuggccuacaaagucccaguucucggcccccg

SEQ ID NO: 65 - RNA, 21 nt, Homo Sapiense
uucacaguggcuaaguucugc

SEQ ID NO: 66 - RNA, 97 nt, Homo Sapiense
accucucuaacaaggugcagagcuuagcugauuggugaacagugauugguuuccgcuuuguucacaguggcuaaguucugcaccugaagagaaggug

SEQ ID NO: 67 - RNA, 22 nt, Homo Sapiense
aacauucauugcugucgguggg

SEQ ID NO: 68 - RNA, 110 nt, Homo Sapiense
ccugugcagagauuauuuuuuaaaaggucacaaucaacauucauugcugucgguggguugaacuguguggacaagcucacugaacaaugaaugcaacuguggccccgcuu

SEQ ID NO: 69 - RNA, 22 nt, Homo Sapiense
uaacacugucugguaaagaugg

SEQ ID NO: 70 - RNA, 95 nt, Homo Sapiense
cggccggcccuggguccaucuuccaguacaguguuggauggucuaauugugaagcuccuaacacugucugguaaagauggcucccggguggguuc

SEQ ID NO: 71 - RNA, 24 nt, Homo Sapiense
aacuggcccucaaagucccgcuuu

SEQ ID NO: 72 - RNA, 83 nt, Homo Sapiense
guggucucagaaucgggguuuugagggcgagaugaguuuauguuuuauccaacuggcccucaaagucccgcuuuuggggucau

SEQ ID NO: 73 - RNA, 23 nt, Homo Sapiense
gaagugcuucgauuuuggggugu

SEQ ID NO: 74 - RNA, 69 nt, Homo Sapiense
gggauacucaaaaugggggcgcuuuccuuuuugucuguacugggaagugcuucgauuuugggguguccc

SEQ ID NO: 75 - RNA, 21 nt, Homo Sapiense
uaaagugcugacagugcagau

SEQ ID NO: 76 - RNA, 82 nt, Homo Sapiense
ccugccggggcuaaagugcugacagugcagauagugguccucuccgugcuaccgcacuguggguacuugcugcuccagcagg

SEQ ID NO: 77 - RNA, 17 nt, Homo Sapiense
agcugguguugugaauc

SEQ ID NO: 78 - RNA, 99 nt, Homo Sapiense
cccuggcauggugugguggggcagcugguguugugaaucaggccguugccaaucagagaacggcuacuucacaacaccagggccacaccacacuacagg

SEQ ID NO: 79 - RNA, 22 nt, Homo Sapiense
uacccuguagaaccgaauuugu

SEQ ID NO: 80 - RNA, 110 nt, Homo Sapiense
ccagagguuguaacguugucuauauauacccuguagaaccgaauuugugugguauccguauagucacagauucgauucuaggggaauauauggucgaugcaaaaacuuca

SEQ ID NO: 81 - RNA, 22 nt, Homo Sapiense
uuuguucguucggcucgcguga

SEQ ID NO: 82 - RNA, 64 nt, Homo Sapiense
ccccgcgacgagccccucgcacaaaccggaccugagcguuuuguucguucggcucgcgugaggc

SEQ ID NO: 83 - RNA, 22 nt, Homo Sapiense
aacccguagauccgaucuugug

SEQ ID NO: 84 - RNA, 81 nt, Homo Sapiense
cccauuggcauaaacccguagauccgaucuuguggugaaguggaccgcacaagcucgcuucuaugggucugugucagugug

SEQ ID NO: 85 - RNA, 21 nt, Homo Sapiense
ugcugacuccuaguccagggc

SEQ ID NO: 86 - RNA, 98 nt, Homo Sapiense
acccaaacccuaggucugcugacuccuaguccagggcucgugauggcuggugggcccugaacgaggggucuggaggccuggguuugaauaucgacagc

SEQ ID NO: 87 - RNA, 20 nt, Homo Sapiense
uagcaccauuugaaaucggu

SEQ ID NO: 88 - RNA, 88 nt, Homo Sapiense
aucucuuacacaggcugaccgauuucuccugguguucagagucuguuuuugucuagcaccauuugaaaucgguuaugauguaggggga

SEQ ID NO: 89 - RNA, 22 nt, Homo Sapiense
uuuggcaaugguagaacucaca

SEQ ID NO: 90 - RNA, 110 nt, Homo Sapiense
gagcugcuugccuccccccguuuuuggcaaugguagaacucacacuggugagguaacaggauccggugguucuagacuugccaacuauggggcgaggacucagccggcac

SEQ ID NO: 91 - RNA, 23 nt, Homo Sapiense
uggcagugucuuagcugguuguu

SEQ ID NO: 92 - RNA, 110 nt, Homo Sapiense
ggccagcugugaguguuucuuuggcagugucuuagcugguuguugugagcaauaguaaggaagcaaucagcaaguauacugcccuagaagugcugcacguuguggggccc

SEQ ID NO: 93 - RNA, 22 nt, Homo Sapiense
ucagugcaucacagaacuuugu

SEQ ID NO: 94 - RNA, 99 nt, Homo Sapiense
caagcacgauuagcauuugaggugaaguucuguuauacacucaggcuguggcucucugaaagucagugcaucacagaacuuugucucgaaagcuuucua

SEQ ID NO: 95 - RNA, 23 nt, Homo Sapiense
aacauucaacgcugucggugagu

SEQ ID NO: 96 - RNA, 110 nt, Homo Sapiense
ugaguuuugagguugcuucagugaacauucaacgcugucggugaguuuggaauuaaaaucaaaaccaucgaccguugauuguacccuauggcuaaccaucaucuacucca

SEQ ID NO: 97 - RNA, 22 nt, Homo Sapiense
uugguccccuucaaccagcugu

SEQ ID NO: 98 - RNA, 88 nt, Homo Sapiense
acaaugcuuugcuagagcugguaaaauggaaccaaaucgccucuucaauggauuugguccccuucaaccagcuguagcuaugcauuga

SEQ ID NO: 99 - RNA, 22 nt, Homo Sapiense
ugagaugaagcacuguagcuca

SEQ ID NO: 100 - RNA, 106 nt, Homo Sapiense
gcgcagcgcccugucucccagccugaggugcagugcugcaucucuggucaguugggagucugagaugaagcacuguagcucaggaagagagaaguuguucugcagc

SEQ ID NO: 101 - RNA, 22 nt, Homo Sapiense
ucucccaacccuuguaccagug

SEQ ID NO: 102 - RNA, 84 nt, Homo Sapiense
cuccccauggcccugucucccaacccuuguaccagugcugggcucagacccugguacaggccugggggacagggaccuggggac

SEQ ID NO: 103 - RNA, 22 nt, Homo Sapiense
ugagguaguagguuguauaguu

SEQ ID NO: 104 - RNA, 22 nt, Homo Sapiense
ugagguaguagguugugugguu

SEQ ID NO: 105 - RNA, 22 nt, Homo Sapiense
ugagguaguagguuguaugguu

SEQ ID NO: 106 - RNA, 22 nt, Homo Sapiense
agagguaguagguugcauaguu

SEQ ID NO: 107 - RNA, 22 nt, Homo Sapiense
ugagguaguagauuguauaguu

SEQ ID NO: 108 - RNA, 22 nt, Homo Sapiense
ugagguaguaguuuguacaguu

SEQ ID NO: 109 - RNA, 22 nt, Homo Sapiense
ugagguaguaaguuguauuguu

SEQ ID NO: 110 - RNA, 23 nt, Homo Sapiense
caaagugcuuacagugcagguag

SEQ ID NO: 111 - RNA, 23 nt, Homo Sapiense
uaaagugcuuauagugcagguag

SEQ ID NO: 112 - RNA, 23 nt, Homo Sapiense
caaagugcucauagugcagguag

SEQ ID NO: 113 - RNA, 22 nt, Homo Sapiense
caaagugccucccuuuagagug

SEQ ID NO: 114 - RNA, 22 nt, Homo Sapiense
gaaagugcuuccuuuuagaggc

SEQ ID NO: 115 - RNA, 23 nt, Homo Sapiense
caaagugcuguucgugcagguag

SEQ ID NO: 116 - RNA, 23 nt, Homo Sapiense
uacccuguagauccgaauuugug

SEQ ID NO: 117 - RNA, 21 nt, Homo Sapiense
uaaggcacccuucugaguaga

SEQ ID NO: 118 - RNA, 22 nt, Homo Sapiense
cagugcaaugaugaaagggcau

SEQ ID NO: 119 - RNA, 23 nt, Homo Sapiense
cagugcaauaguauugucaaagc

SEQ ID NO: 120 - RNA, 23 nt, Homo Sapiense
cagugcaaugauauugucaaagc

SEQ ID NO: 121 - RNA, 23 nt, Homo Sapiense
uagugcaauauugcuuauagggu

SEQ ID NO: 122 - RNA, 22 nt, Homo Sapiense
ugagaacugaauuccauaggcu

SEQ ID NO: 123 - RNA, 22 nt, Homo Sapiense
ucagugcacuacagaacuuugu

SEQ ID NO: 124 - RNA, 22 nt, Homo Sapiense
aacauucaaccugucggugagu

SEQ ID NO: 125 - RNA, 23 nt, Homo Sapiense
aacauucauuguugucggugggu

SEQ ID NO: 126 - RNA, 21 nt, Homo Sapiense
augaccuaugaauugacagac

SEQ ID NO: 127 - RNA, 22 nt, Homo Sapiense
uagguaguuuccuguuguuggg

SEQ ID NO: 128 - RNA, 23 nt, Homo Sapiense
ugugcaaaucuaugcaaaacuga

SEQ ID NO: 129 - RNA, 22 nt, Homo Sapiense
uaauacugccugguaaugauga

SEQ ID NO: 130 - RNA, 22 nt, Homo Sapiense
uaauacugucugguaaaaccgu

SEQ ID NO: 131 - RNA, 22 nt, Homo Sapiense
gagcuuauucauaaaagugcag

SEQ ID NO: 132 - RNA, 21 nt, Homo Sapiense
uucacaguggcuaaguuccgc

SEQ ID NO: 133 - RNA, 23 nt, Homo Sapiense
aggcaguguaguuagcugauugc

SEQ ID NO: 134 - RNA, 22 nt, Homo Sapiense
uggcaguguauuguuagcuggu

SEQ ID NO: 135 - RNA, 22 nt, Homo Sapiense
aggcaguguauuguuagcuggc

SEQ ID NO: 136 - RNA, 22 nt, Homo Sapiense
uauugcacauuacuaaguugca

SEQ ID NO: 137 - RNA, 22 nt, Homo Sapiense
aauugcacuuuagcaaugguga

SEQ ID NO: 138 - RNA, 22 nt, Homo Sapiense
uauugcacuugucccggccugu

SEQ ID NO: 139 - RNA, 23 nt, Homo Sapiense
uaagugcuuccauguuuugguga

SEQ ID NO: 140 - RNA, 23 nt, Homo Sapiense
uaagugcuuccauguuuuaguag

SEQ ID NO: 141 - RNA, 23 nt, Homo Sapiense
uaagugcuuccauguuucagugg

SEQ ID NO: 142 - RNA, 23 nt, Homo Sapiense
uaagugcuuccauguuugagugu

SEQ ID NO: 143 - RNA, 22 nt, Homo Sapiense
aaagugcuucccuuuggacugu

SEQ ID NO: 144 - RNA, 21 nt, Homo Sapiense
aaagugcuuccuuuuagaggg

SEQ ID NO: 145 - RNA, 22 nt, Homo Sapiense
aaagugcuuccuuuuagagggu

SEQ ID NO: 146 - RNA, 22 nt, Homo Sapiense
aaagugcuucucuuuggugggu

SEQ ID NO: 147 - RNA, 21 nt, Homo Sapiense
aaagugcuuccuuuuugaggg

SEQ ID NO: 148 - RNA, 22 nt, Homo Sapiense
cauugcacuugucucggucuga

SEQ ID NO: 149 - RNA, 22 nt, Homo Sapiense
aacccguagauccgaacuugug

SEQ ID NO: 150 - RNA, 22 nt, Homo Sapiense
cacccguagaaccgaccuugcg

SEQ ID NO: 151 - RNA, 22 nt, Homo Sapiense
caagcucgugucuguggguccg

SEQ ID NO: 152 - RNA, 21 nt, Homo Sapiense
aggcggagacuugggcaauug

SEQ ID NO: 153 - RNA, 22 nt, Homo Sapiense
acugcugagcuagcacuucccg

SEQ ID NO: 154 - RNA, 22 nt, Homo Sapiense
cgucuuacccagcaguguuugg

SEQ ID NO: 155 - RNA, 22 nt, Homo Sapiense
ggugcagugcugcaucucuggu

SEQ ID NO: 156 - RNA, 22 nt, Homo Sapiense
accaucgaccguugauuguacc

SEQ ID NO: 157 - RNA, 22 nt, Homo Sapiense
accacugaccguugacuguacc

SEQ ID NO: 158 - RNA, 22 nt, Homo Sapiense
uauggcacugguagaauucacu

SEQ ID NO: 159 - RNA, 22 nt, Homo Sapiense
gugaauuaccgaagggccauaa

SEQ ID NO: 160 - RNA, 23 nt, Homo Sapiense
uuuggcacuagcacauuuuugcu

SEQ ID NO: 161 - RNA, 22 nt, Homo Sapiense
aaucaugugcagugccaauaug

SEQ ID NO: 162 - RNA, 22 nt, Homo Sapiense
ccaguggggcugcuguuaucug

SEQ ID NO: 163 - RNA, 22 nt, Homo Sapiense
uaaugccccuaaaaauccuuau

SEQ ID NO: 164 - RNA, 22 nt, Homo Sapiense
cugccaauuccauaggucacag

SEQ ID NO: 165 - RNA, 23 nt, Homo Sapiense
aaaagugcuuacagugcagguag

SEQ ID NO: 166 - RNA, 22 nt, Homo Sapiense
cugcaauguaagcacuucuuac

SEQ ID NO: 167 - RNA, 22 nt, Homo Sapiense
acugcagugaaggcacuuguag

SEQ ID NO: 168 - RNA, 23 nt, Homo Sapiense
uaaggugcaucuagugcagauag

SEQ ID NO: 169 - RNA, 23 nt, Homo Sapiense
acugcccuaagugcuccuucugg

SEQ ID NO: 170 - RNA, 23 nt, Homo Sapiense
uaaggugcaucuagugcaguuag

SEQ ID NO: 171 - RNA, 22 nt, Homo Sapiense
ugcccuaaaugccccuucuggc

SEQ ID NO: 172 - RNA, 22 nt, Homo Sapiense
aguuuugcauaguugcacuaca

SEQ ID NO: 173 - RNA, 22 nt, Homo Sapiense
acuguaguaugggcacuuccag

SEQ ID NO: 174 - RNA, 22 nt, Homo Sapiense
cggguggaucacgaugcaauuu

SEQ ID NO: 175 - RNA, 23 nt, Homo Sapiense
agguugggaucgguugcaaugcu

SEQ ID NO: 176 - RNA, 22 nt, Homo Sapiense
ggguggggauuuguugcauuac

SEQ ID NO: 177 - RNA, 22 nt, Homo Sapiense
caucuuacugggcagcauugga

SEQ ID NO: 178 - RNA, 22 nt, Homo Sapiense
caucuuccaguacaguguugga

SEQ ID NO: 179 - RNA, 22 nt, Homo Sapiense
acaguagucugcacauugguua

SEQ ID NO: 180 - RNA, 23 nt, Homo Sapiense
cccaguguucagacuaccuguuc

SEQ ID NO: 181 - RNA, 21 nt, Homo Sapiense
aucacauugccagggauuacc

SEQ ID NO: 182 - RNA, 22 nt, Homo Sapiense
uggguuccuggcaugcugauuu

SEQ ID NO: 183 - RNA, 22 nt, Homo Sapiense
uggcucaguucagcaggaacag

SEQ ID NO: 184 - RNA, 22 nt, Homo Sapiense
ugccuacugagcugauaucagu

SEQ ID NO: 185 - RNA, 24 nt, Homo Sapiense
gcugguuucauauggugguuuaga

SEQ ID NO: 186 - RNA, 22 nt, Homo Sapiense
acugauuucuuuugguguucag

SEQ ID NO: 187 - RNA, 22 nt, Homo Sapiense
ugaccgauuucuccugguguuc

SEQ ID NO: 188 - RNA, 22 nt, Homo Sapiense
cugguuucacaugguggcuuag

SEQ ID NO: 189 - RNA, 22 nt, Homo Sapiense
aaucacuaaccacacggccagg

SEQ ID NO: 190 - RNA, 22 nt, Homo Sapiense
aguuuugcagguuugcauuuca

SEQ ID NO: 191 - RNA, 23 nt, Homo Sapiense
aagugccgccaucuuuugagugu

SEQ ID NO: 192 - RNA, 20 nt, Homo Sapiense
acucaaacugugggggcacu

SEQ ID NO: 193 - RNA, 22 nt, Homo Sapiense
acucaaaaugggggcgcuuucc

SEQ ID NO: 194 - RNA, 22 nt, Homo Sapiense
ugugacugguugaccagagggg

SEQ ID NO: 195 - RNA, 22 nt, Homo Sapiense
uagguuauccguguugccuucg

SEQ ID NO: 196 - RNA, 22 nt, Homo Sapiense
aaucauacacgguugaccuauu

SEQ ID NO: 197 - RNA, 22 nt, Homo Sapiense
aucacacaaaggcaacuuuugu

SEQ ID NO: 198 - RNA, 22 nt, Homo Sapiense
agagguugcccuuggugaauuc

SEQ ID NO: 199 - RNA, 22 nt, Homo Sapiense
uauacaagggcaagcucucugu

SEQ ID NO: 200 - RNA, 23 nt, Homo Sapiense
agguuguccguggugaguucgca

SEQ ID NO: 201 - RNA, 22 nt, Homo Sapiense
gucauacacggcucuccucucu

SEQ ID NO: 202 - RNA, 22 nt, Homo Sapiense
agaggcuggccgugaugaauuc

SEQ ID NO: 203 - RNA, 22 nt, Homo Sapiense
aaucauacagggacauccaguu

SEQ ID NO: 204 - RNA, 22 nt, Homo Sapiense
aaucguacagggucauccacuu

SEQ ID NO: 205 - RNA, 22 nt, Homo Sapiense
ugaguauuacauggccaaucuc

SEQ ID NO: 206 - RNA, 22 nt, Homo Sapiense
ggagaaauuauccuuggugugu

SEQ ID NO: 207 - RNA, 22 nt, Homo Sapiense
auucugcauuuuuagcaaguuc

SEQ ID NO: 208 - RNA, 22 nt, Homo Sapiense
auaauacaugguuaaccucuuu

SEQ ID NO: 209 - RNA, 23 nt, Homo Sapiense
ugucacucggcucggcccacuac

SEQ ID NO: 210 - RNA, 21 nt, Homo Sapiense
uuaauaucggacaaccauugu

SEQ ID NO: 211 - RNA, 22 nt, Homo Sapiense
uacugcagacguggcaaucaug

SEQ ID NO: 212 - RNA, 22 nt, Homo Sapiense
ugauugguacgucuguggguag

SEQ ID NO: 213 - RNA, 21 nt, Homo Sapiense
uacugcagacaguggcaauca

SEQ ID NO: 214 - RNA, 23 nt, Homo Sapiense
ugugcaaauccaugcaaaacuga

SEQ ID NO: 215 - RNA, 23 nt, Homo Sapiense
aguuuugcagguuugcauccagc

SEQ ID NO: 216 - RNA, 22 nt, Homo Sapiense
acugcauuaugagcacuuaaag

SEQ ID NO: 217 - RNA, 22 nt, Homo Sapiense
uagaguuacacccugggaguua

SEQ ID NO: 218 - RNA, 22 nt, Homo Sapiense
aacuggccuacaaagucccagu

SEQ ID NO: 219 - RNA, 22 nt, Homo Sapiense
acaggugagguucuugggagcc

SEQ ID NO: 220 - RNA, 24 nt, Homo Sapiense
ucccugagacccuuuaaccuguga